(19) United States
(12) Patent Application Publication
(10) Pub. No.: US 2003/0139609 A1
(43) Pub. Date: Jul. 24, 2003

(54) Aziridinyl quinone antitumor agents based on indoles and cyclopent[b]indoles

(76) Inventors: Edward B. Skibo, Mesa, AZ (US); Chengguo Xing, Somerville, MA (US)

Correspondence Address: FENNEMORE CRAIG, 3003 NORTH CENTRAL AVENUE, SUITE 2600, PHOENIX, AZ, 85012, US

(21) Appl. No.: 10/241,135

(22) Filed: Sep. 10, 2002

Related U.S. Application Data
(60) Provisional application No. 60/318,846, filed on Sep. 10, 2001.

(51) Int. Cl. A61K 31/404; A61K 31/403; C 07D 4 3/02

(52) U.S. Cl. 548/427; 548/465; 514/411; 514/414

(57) ABSTRACT

A large number of aziridinyl quinones represented by Series 1-9 were studied with respect to their DT-diaphorase substrate activity, DNA reductive alkylation, cytostatic/cytotoxic activity, and in vivo activity. As a result generalizations have been made with respect with respect to the following: DT-diaphorase substrate design, DT-diaphorase-cytotoxicity QSAR, and DNA reductive alkylating agent design. A saturating relationship exists between the substrate specificity for human recombinant DT-diaphorase and the cytotoxicity in the human H460 non-small-cell lung cancer cell line. The interpretation of this relationship is that reductive activation is no longer rate limiting for substrates with high DT-diaphorase substrate specificities. High DT-diaphorase substrate specificity is not desirable in the indole and cylopent[b]indole systems because of the result is the loss of cancer selectivity along with increased toxicity. We conclude that aziridinyl quinones of this type should possess a substrate specificity (VMAX/KM )<10×10-4 s-1 for DT-diaphorase in order not to be too toxic or nonselective. While some DNA alkylation was required for cytostatic and cytotoxic activity by Series 1-9, too much alkylation results in loss of cancer selectivity as well as increased in vivo toxicity. Indeed, the most lethal compounds are the indole systems with a leaving group in the 3a-position (like the antitumor agent EO-9). We conclude that relatively poor DNA alkylating agents (according to our assay) show the lowest toxicity with the highest antitumor activity.

### TECHNICAL FIELD

This application claims the benefit of U.S. Provisional Patent Application Serial No. 60/318,846 filed Sep. 10, 2001.

Financial assistance for this project was provided by the US government through the National Science Foundation under Grant Number CHE-9522640, through the National Institutes of Health under Grant Number CA73758, and through the Arizona Disease Control Commission, The United States Government may own certain rights to this invention.

### INTRODUCTION

The present invention relates generally to the field of chemotherapy and more particularly to the isolation, elucidation of novel antineoplastic agents based on indoles and cyclopent[b]indoles.

### BACKROUND OF THE INVENTION

Many of the clinically used antitumor agents that have withstood the test of time are DNA-directed alkylating agents utilizing the aziridine alkylating center. Noteworthy examples include the nitrogen mustards, {Struck, R. F., Nitrogen Mustard and Related Structures, 1995, 112-120}nitrosoureas, {Elliott, R. D., Nitrosoureas, 1995, 134-143} thiotepa,{Reynolds, R. C., Aziridines, 1995, 187-197} AZQ, {Reynolds, R. C., Aziridines, 1995, 187-197} triethylenemelamine {Reynolds, R. C., Aziridines, 1995, 187-197} and mitomycin C {Tomasz, M. and Palom, Y., The mitomycin bioreductive antitumor agents: Cross-linking and alkylation of DNA as the molecular basis of their activity, Pharmacol. Ther., 1997, 76, 73-87}. These compounds have been thoroughly studied and have been subjected to intense analogue development for over forty years. Examples of aziridinyl quinone antitumor agents developed recently are shown in Chart 1.

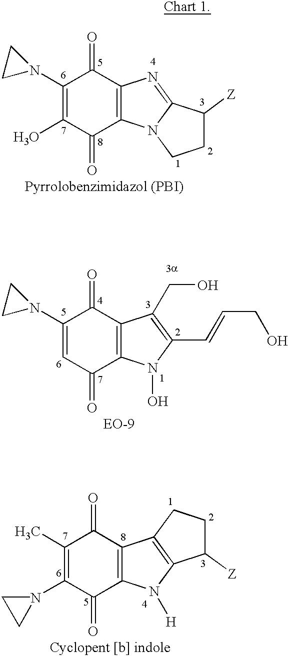

The PBIs were developed in this laboratory and found to possess cytotoxicity with minimal antitumor activity. {Skibo, E. B., The Discovery of the Pyrrolo[1,2-a]benzimidazole Antitumor Agents—The Design of Selective Antitumor Agents, Curr. Med. Chem., 1996, 2, 900-931; Skibo, E. B., Pyrrolobenzimidazoles in Cancer Treatment, Exper. Opin. Ther. Patents, 1998, 8, 673-701} The indoloquinone EO9 was considered to be a promising antitumor agent, {Hendriks, H. R., Pizao, P. E., Berger, D. P., Kooistra, K. L., Bibby, M. C., Boven, E., Dreef-van der Meulen, H. C., Henrar, H. H., Fiebig, H. H., Double, J. A., Hornstra, H. W., Pinedo, H. M., Workman, P., and Schwartsmann, G., EO9: A Novel Bioreductive Alkylating Indoloquinone With Prefential Solid Tumour Activity and Lack of Bone Marrow Toxicity in Preclinical Models, Eur. J. Cancer, 1993, 29A, 8997-906; Maliepaard, M., Wolfs, A., Groot, S. E., de Mol, N. J., and Janssen, L. H. M., Indoloquinone EO9: DNA Interstrand Cross-linking Upon Reduction by DT-Diaphorase or Xanthine Oxidase, Br .J. Cancer, 1995, 71, 836-839}but Phase I clinical trials revealed short plasma half-lives as well as toxicity. {Schellens, J. H. M., Planting, A. S. T., Vanacker, B. A. C., Loos, W. J., Deboerdennert, M., Vanderburg, M. E. L., Koier, I., Krediet, R. T., Stoter, G., and Verweij, J., Phase-I and Pharmacological Study of the Novel Indoloquinone Bioreductive Alkylating Cytotoxic Drug E09, J. Nat. Cancer Inst., 1994, 86, 906-912; Aamdal, S., Lund, B., Koier, I., Houten, M., Wanders, J., and Verweij, J., Phase I trial with Weekly EO9, a Novel Bioreductive Alkylating Indoloquinone, by the EORTC Early Clinical Study Group (ECSG), Cancer Chemother. Pharmacol, 2000, 45, 85-88; Loadman, P. M., Phillips, R. M., Lim, L. E., and Bibby, M. C., Pharmacological Properties of a New Aziridinylbenzoquinone, RH1 (2,5-diaziridinyl-3-(Hydroxymethyl)-6-methyl-1,4-benzoquinone), in Mice, Biochem Pharmaco, 2000, 59, 831-837.} The cyclopent[b]indoles, reported in the past year, {Xing, C. G., Wu, P., Skibo, E. B., and Dorr, R. T., Design of cancer-specific antitumor agents based on aziridlinylcyclopent[b]indoloquinones, J. Med. Chem., 2000, 43, 457-466 } possess promising antitumor activity.

### SUMMARY OF THE INVENTION

It has been known that the level of the enzyme DT-Diaphorase is elevated in certain types of cancer tissue, and that the enzyme may be provided with a chemical substrate, which substrate will result in the production of certain reduction products which have a cytotoxic effect. Thus, by providing an effective chemical substrate, as disclosed herein, to an organism affected with certain types of cancer tissue, the cancerous tissue may be destroyed while leaving non-cancerous tissue substantially unharmed.

More specifically, the present invention involves a discovery by the inventors that an antitumor agent's substrate specificity for the enzyme DT-Diaphorase is related to the selectivity for histological cancer types. Thus, “poor” substrates, defined as possessing a substrate specificity (VMAX/KM )<10×10-4 s-1, are highly selective for histological cancer types possessing high levels of DT-Diaphorase. In contrast, “excellent” substrates, defined as possessing a substrate specificity (VMAX/KM )>10×10-4 s-1, possess poor selectivity for histological cancer types and are toxic to tissues regardless of the levels of DT-Diaphorase.

Disclosed herein is a comprehensive structure-activity study of cyclopent[b]indole-based (Series 1-5) and indole-based (Series 6-9) aziridinyl quinones represented by the nine general structures shown in Chart 2. Diverse structures were generated by varying the position of the aziridinyl and quinone methyl groups (if present) as well as the indole N-substituent. These structural features can influence the interaction of quinone antitumor agents with DNA, {Zhou, R. and Skibo, E. B., Chemistry of the Pyrrolo[1,2-a]benzimidazole Antitumor Agents: Influence of the 7-Substituent on the Ability to Alkylate DNA and Inhibit Topoisomerase II., J. Med. Chem., 1996, 39, 4321-4331; Xing, C. G., Wu, P., Skibo, E. B., and Dorr, R. T., Design of cancer-specific antitumor agents based on aziridlinylcyclopent[b]indoloquinones, J. Med. Chem., 2000, 43, 457-466} and this study would clarify the structural requirements for such binding. The structure of EO9 {Beall, H. D., Hudnott, A. R., Winski, S., Siegel, D., Swann, E., Ross, D., and Moody, C. J., Indolequinone antitumor agents: Relationship between quinone structure and rate of metabolism by recombinant human NQO1, Bioorg .Medicinal Chem. Letter., 1998, 8, 545-548; Naylor, M. A., Swann, E., Everett, S. A., Jaffar, M., Nolan, J., Robertson, N., Lockyer, S. D., Patel, K. B., Dennis, M. F., Stratford, M. R. L., Wardman, P., Adams, G. E., Moody, C. J., and Stratford, I. J., Indolequinone antitumor agents: Reductive activation and elimination from (5-methoxy-1-methyl-4,7-dioxoindol-3-yl) methyl derivatives and hypoxia-selective cytotoxicity in vitro, J. Med. Chem., 1998, 41, 2720-2731; Beall, H. D., Winski, S., Swann, E., Hudnott, A. R., Cotterill, A. S., OSullivan, N., Green, S. J., Bien, R., Siegel, D., Ross, D., and Moody, C. J., Indolequinone antitumor agents: Correlation between quinone structure, rate of metabolism by recombinant human NAD(P)H:quinone oxidoreductase, and in vitro cytotoxicity, J. Med. Chem., 1998, 41, 4755-4766; Phillips, R. M., Naylor, M. A., Jaffar, M., Doughty, S. W., Everett, S. A., Breen, A. G., Choudry, G. A., and Stratford, I. J., Bioreductive activation of a series of indolequinones by human DT-diaphorase: Structure-activity relationships, J. Med. Chem., 1999, 42, 4071-4080} inspired the synthesis of indole-based diol derivatives and their acetylated analogues.

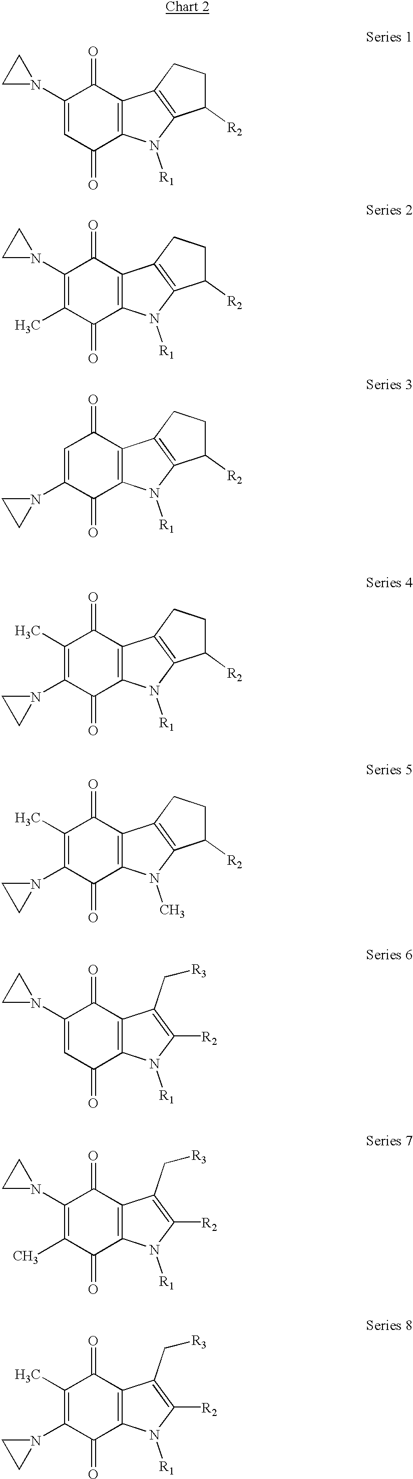

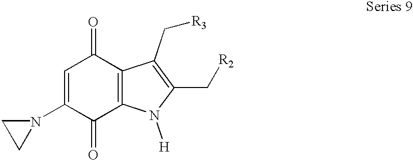

The study of a large number of compounds required use of a chemical/biochemical prescreen that could predict biological activity. The inventors successfully used the quinone substrate specificity (V_{MAX}/K_{M}) for human DT-diaphorase and the percent DNA alkylation to assess cytotoxic and antitumor capability. From this study, we were able to elucidate structure activity relationships valuable in antitumor design.

### BRIEF DESCRIPTION OF THE DRAWINGS

FIG. 1A. Model A. The active sites of human DT-diaphorase from NCI-H460 non-small-lung cancer obtained from the protein database (1D4A). Important amino acid residues are labeled.

FIG. 1B. Model B. The S-amino PBI substrate in the DT-diaphorase from NCI-H460 non-small-lung cancer.

FIG. 2. Model of an N-unsubstituted cyclopent[b]indole hydroquinone in the major groove at a GC base pair with the aziridinyl group reacted with a phosphate. The 3-substituent is a hydrogen bond acceptor of the cytosine amino NH, length is 2.21 Å. The indole NH is a hydrogen bond donor to the guanine 6-carbonyl or guanine N(7), lengths are 2.57 and 1.86 Å respectively. The hydroquinone hydroxyl is a hydrogen bond donor to the guanine N(7), length is 1.86 Å.

FIG. 3. Plot of −LogLC50 for Series 1-9 compounds in NCI-H460 non-small-lung cancer cell lines versus the specificity for recombinant DT-diaphorase from the same cell line. The solid line was generated from equation 1

FIG. 4. Bar graph of −Log of TGI, GI50, and LC50 for 5a and 3a versus histological cancer type. The −Log parameter values are the average of 6-8 cell lines within each histological cancer.

### DESCRIPTION OF PREFERRED EMBODIMENTS

Synthesis. The preparation of Series 1-9 was carried out as outlined in Schemes 1-6. Many of the synthetic procedures utilized for these preparations are straightforward and are briefly outlined below.

Shown in schemes 1-3 are the synthetic methodologies for the preparation of the cyclopent[b]indoles (Series 1-5). Preparation of the substituted cyclopent[b]indol-3-one system was carried out by the Fischer Indole reaction as previously described. {Xing, C. G., Wu, P., Skibo, E. B., and Dorr, R. T., Design of cancer-specific antitumor agents based on aziridlinylcyclopent[b]indoloquinones, J. Med. Chem., 2000, 43, 457-466}Elaboration of the quinone functional group was carried out by the nitration, catalytic reduction of the nitro group, and finally Fremy oxidation. {Zimmer, H., Lankin, D. C., and Horgan, S. W., Oxidations with Potassium Nitrosodisulfonate (Fremy's Radical). The Teuber Reaction, Chem. Rev., 1971, 71, 229-246; Skibo, E. B., Islam, I., Schulz, W. G., Zhou, R., Bess, L., and Boruah, R., The Organic Chemistry of the Pyrrolo[1,2-a]benzimidazole Antitumor Agents. An Example of Rational Drug Design. Synlett, 1996, 297-309} The 3-hydroxy group (1a-4a series) was obtained by borohydride reduction of the 3-one group. Acetylation of the 3-hydroxy group afforded a mixture of O- and N-acetylated compounds, that constituted the 1b-4b and 1c-4c series. N-Methylation of the cyclopent[b]indol-3-one system, followed by quinone and 3-hydroxyl or 3-acetoxy elaboration, provided series 5.

Shown in schemes 4-6 are the synthetic methodologies for the preparation of the indoles (Series 6-9). Preparation of the 2-ethylcarboxyl indole substituted systems were carried out by the Japp-Klingemann/Fischer Indole reaction as previously described. {Liu, R., Zhang, P., T. Gan, T., and Cook, J. M., Regiospecific Bromination of 3-Methylindoles with NBS and Its Application to the Concise Synthesis of Optically Active UnusualTryptophans Present in Marine Cyclic Peptides, J. Org. Chem., 1997, 62, 7447-7456; Xing, C. G., Wu, P., Skibo, E. B., and Dorr, R. T., Design of Cancer-specific Antitumor agents Based on Aziridlinylcyclopent[b]indoloquinones, J. Med. Chem., 2000, 43, 457-466} Vilsmeier formylation and borohydride reduction afforded the 3-hydroxymethyl derivative of these indole systems. The 2-ethylcarboxyl substituent was either retained or reduced with LAH to the 2-hydroxymethyl group. The hydroxymethyl groups were either left as is or acetylated to afford the acetate leaving group. Finally, quinone elaboration and aziridination were carried out as previously described. {Zimmer, H., Lankin, D. C., and Horgan, S. W., Oxidations with Potassium Nitrosodisulfonate (Fremy's Radical). The Teuber Reaction, Chem. Rev., 1971, 71, 229-246; Skibo, E. B., Islam, I., Schulz, W. G., Zhou, R., Bess, L., and Boruah, R., The Organic Chemistry of the Pyrrolo[1,2-a]benzimidazole Antitumor Agents. An Example of Rational Drug Design. Synlett, 1996, 297-309}

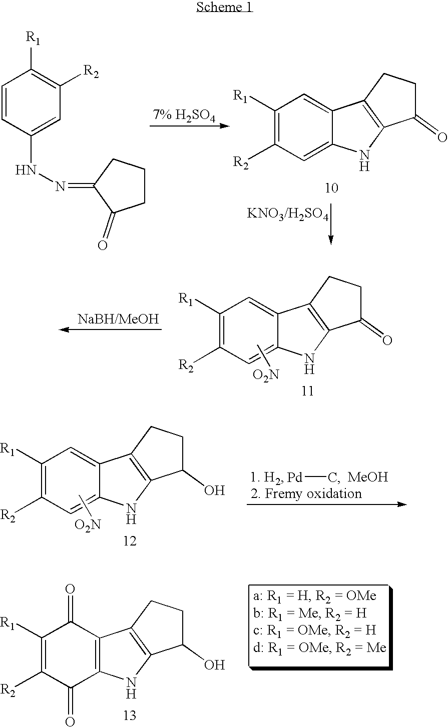

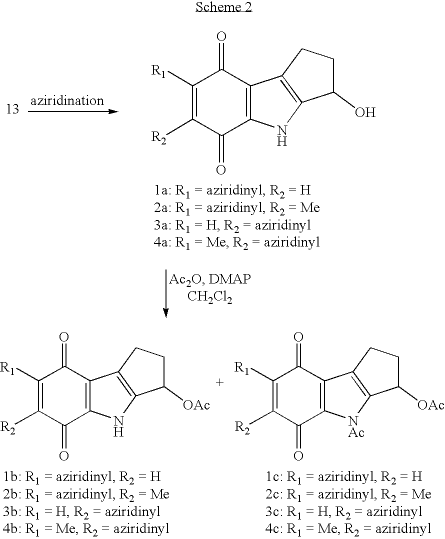

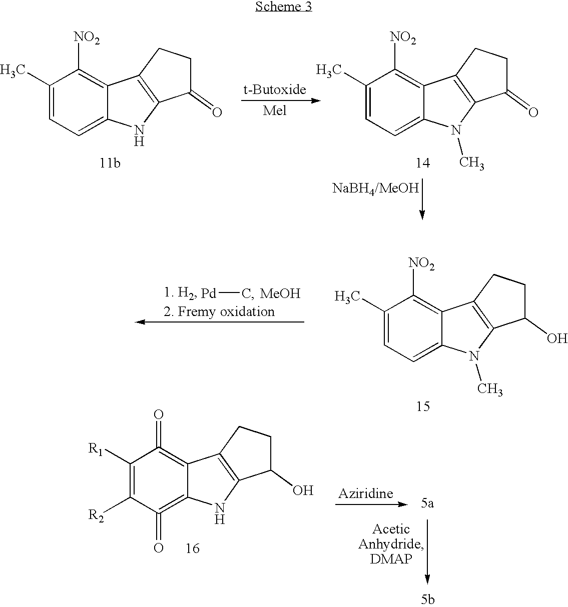

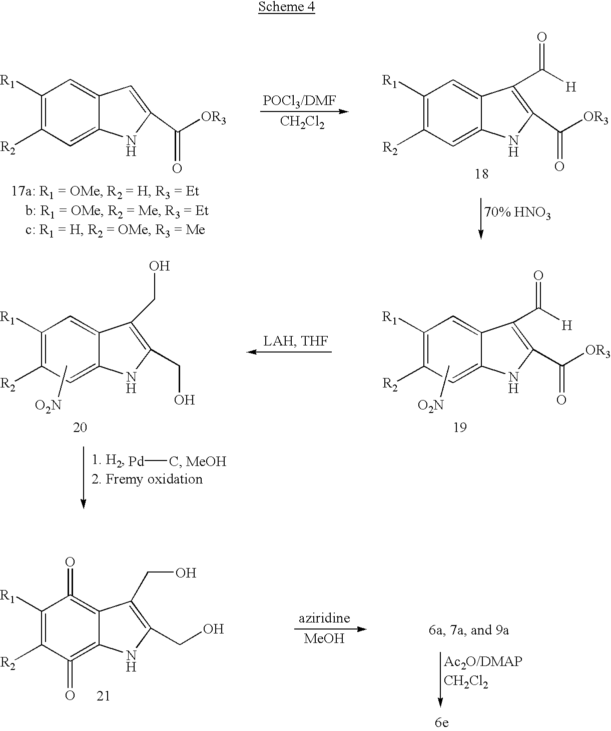

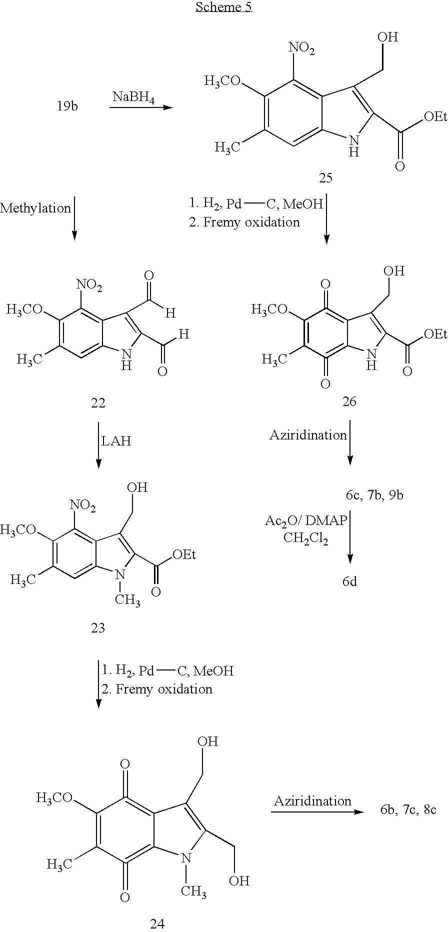

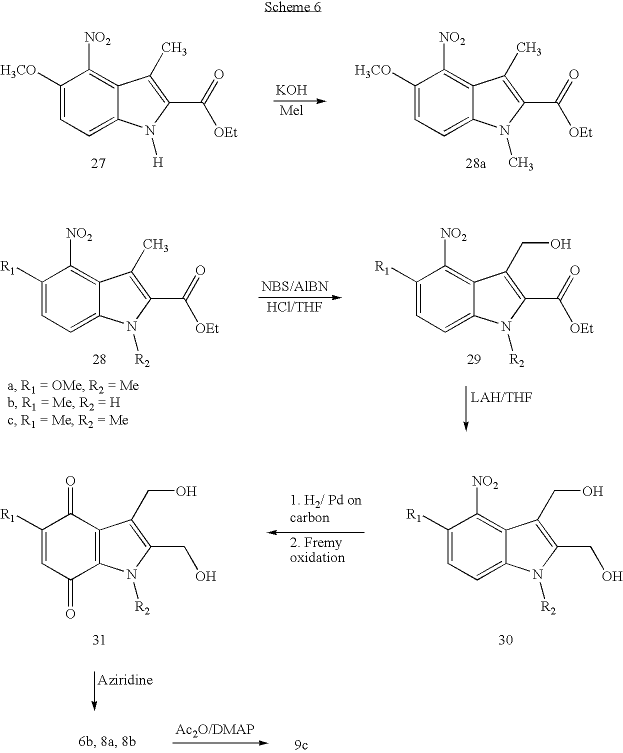

DT-Diaphorase Substrate Screening of Series 1-9. The enzyme DT-diaphorase is an NAD(P)H-dependent reducing enzyme that converts quinones and other substrates to the corresponding two-electron reduction products. The hydroquinone reduction product is cytotoxic because its aziridinyl nitrogen is readily protonated at physiological pH resulting in alkylation reactions, Scheme 7. The phosphate backbone {Schulz, W. G., Nieman, R. A., and Skibo, E. B., Evidence for DNA Phosphate Backbone Alkylation and Cleavage by Pyrrolo[1,2-a]benzimidazoles, Small Molecules Capable of Causing Sequence Specific Phosphodiester Bond Hydrolysis, Proc. Natl. Acad. Sci,. U.S.A., 1995, 92, 11854-11858} and the purine N(7)-position {Hargreaves, R. H. J., Mayalarp, S. P., Butler, J., McAdam, S. R., OHare, C. C., and Hartley, J., A. Cross-linking and sequence specific alkylation of DNA by aziridinyl quinones. 2. Structure requirements for sequence selectivity, J. Med. Chem., 1997, 40, 357-361; Alley, S. C., Brameld, K. A., and Hopkins, P. B., DNA Interstrand Cross-Linking by 2,5-Bis(1-aziridinyl)-1,4-benzoquinone: Nucleotide Sequence Preferences and Covalent Structures of the dG-to-dG Cross-Links at 5′-d(GNₙC) in Synthetic Oligonucleotide Duplexes, J. Am. Chem. Soc., 1994, 116, 2734-2741}of DNA are the usual nucleophile targets. This enzyme is elevated in some cancers and the reduction process leads to activation of antitumor agents specifically in these cancers. {Rauth, A. M., Goldberg, Z., and Misra, V., DT-diaphorase: Possible roles in cancer chemotherapy and carcinogenesis, Oncol. Res., 1997, 9, 339-349; Rauth, A. M., Melo, T., and Misra, V. Bioreductive therapies: An overview of drugs and their mechanisms of action, Int. J. Radiat. Oncol. Biol. Phys., 1998, 42, 755-762; Stratford, I. J. and Workman, P., Bioreductive drugs into the next millennium, Anti. Cancer Drug. Des., 1998, 13, 519-528} The reductive activation process is well known with substrates such as mitomycin C, {Siegel, D., Beall, H., Senekowitsch, C., Kasai, M., Arai, H., Gibson, N. W., and Ross, D., Bioreductive Activation of Mitomycin C by DT-Diaphorase, Biochemistry, 1992, 31, 7879-7889; Cummings, J., Spanswick, V. J., Tomasz, M., and Smyth, J. F., Enzymology of mitomycin C metabolic activation in tumour tissue—Implications for enzyme-directed bioreductive drug development, Biochem. Pharmacol., 1998, 56, 405-414}EO9, {Naylor, M. A., Swann, E., Everett, S. A., Jaffar, M., Nolan, J., Robertson, N., Lockyer, S. D., Patel, K. B., Dennis, M. F., Stratford, M. R. L., Wardman, P., Adams, G. E., Moody, C. J., and Stratford, I. J., Indolequinone antitumor agents: Reductive activation and elimination from (5-methoxy-1-methyl-4,7-dioxoindol-3-yl) methyl derivatives and hypoxia-selective cytotoxicity in vitro, J. Med. Chem., 1998, 41, 2720-2731; Jaffar, M., Naylor, M. A., Robertson, N., and Stratford, I. J,. Targeting hypoxia with a new generation of indolequinones, Anti. Cancer Drug. Des., 1998, 13, 593-609} and the PBIs. {Skibo, E. S., Gordon, S., Bess, L., Boruah, R., and Heileman, J., Studies of Pyrrolo[1,2-a]benzimidazole Quinone DT-Diaphorase Substrate Activity, Topoisomerase II Inhibition Activity, and DNA Reductive Alkylation, J. Med. Chem., 1997, 40, 1327-1339; Skibo, E. B., Pyrrolobenzimidazoles in cancer treatment, Expert. Opin. Ther. Patents, 1998, 8, 673-701}

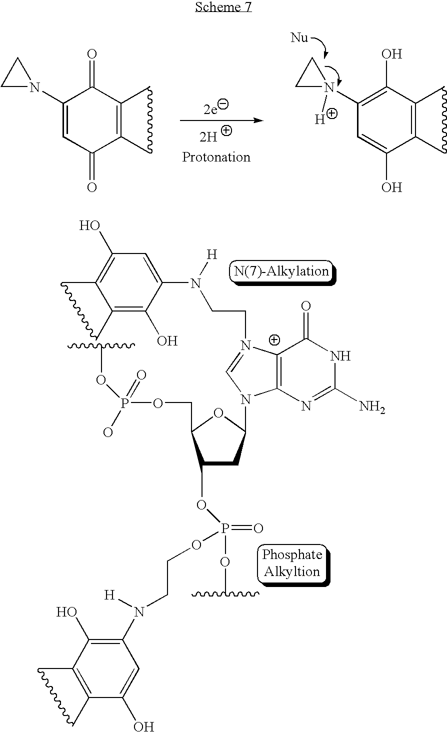

Listed in Tables 1-4 are the human DT-diaphorase kinetic parameters for Series 1-9, along with the DNA alkylation percentages. These parameters include the apparent dissociation constant K_{M}, the apparent maximal velocity V_{MAX}, and the substrate specificity, V_{MAX}/K_{M}. The designation “apparent” refers to the present of productive and nonproductive constants in K_{M} and V_{MAX}. {Fersht, A. R., The Hydrogen Bond in Molecular Recognition Trends, Biochem. Sci., 1987, 12, 301-304} These constants cancel in the ratio V_{MAX}/K_{M}, which is the best parameter to compare substrates. Human DT-diaphorase (from the non-small cell lung NCI-H460 cell line) {Beall, H. D., Mulcahy, R. T., Siegel, D., Traver, R D., Gibson, N. W., and Ross, D., Metabolism of Bioreductive Antitumor Compounds by Purified Rat and Human DT-Diaphorase Cancer Res., 1994, 54, 3196-3201 } was used to obtain these kinetic parameters. This enzyme is the same as that present in other human tissues, cancerous or otherwise.

The human DT-diaphorase enzyme has been cloned and its crystal structure determined (1D4A in the Protein Database). {Faig, M., Bianchet, M. A., Talalay, P., Chen, S., Winski, S., Ross, D., and Amzel, L. M., Structures of Recombinant Human and Mouse NAD(P)H:quinone Oxidoreductases: Species Comparison and Structural Changes with Substrate Binding and Release, Proc. Natl. Acad. Sci. U.S.A., 2000, 97, 3177-82} In a study of the DT-diaphorase reduction of pyrrolo[1,2-a]benzimidazoles (PBIs), the enzyme-bound PBI structure was obtained by superimposition into the human DT-diaphorase active site. {Huang, X., Suleman, A., and Skibo, E. B., Rational Design of Pyrrolo[1,2-a ]benzimidazole, Based Antitumor Agents Targeting the DNA Major Groove Bioorg. Chem., 2000, Accepted Dec. Issue.}

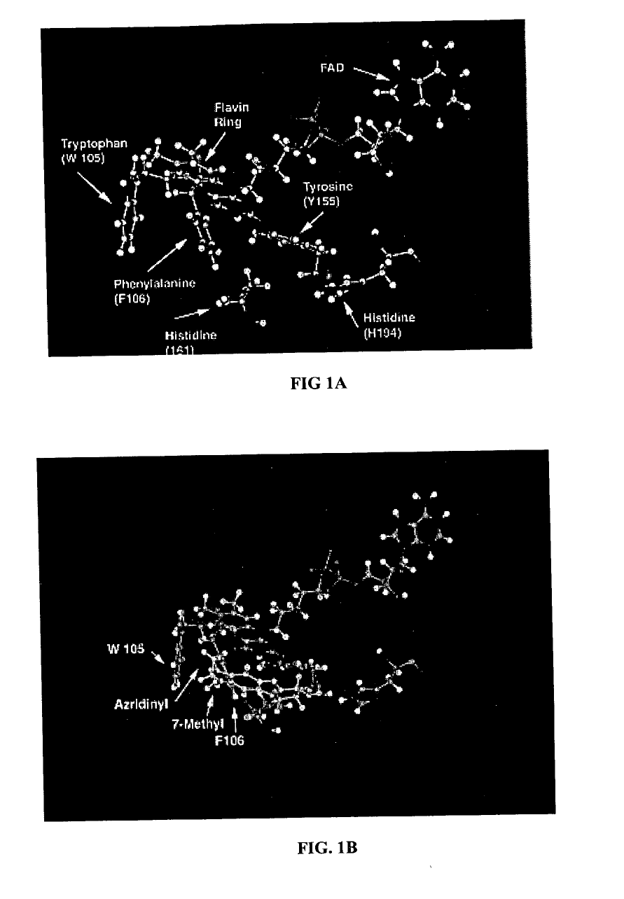

Shown in FIG. 1 is the active site of DT-diaphorase (1D4A) as well as the same active site containing a PBI aziridinyl quinone. Although the structure shown in FIG. 1B not crystallographically determined, it permitted the rationalization of indole and cyclopent[b]indole substrate specificity as illustrated. FIG. 1 illustrates Model A, the active sites of human DT-diaphorase from NCI-H460 non-small-lung cancer obtained from the protein database (1D4A). Important amino acid residues are labeled. Also illustrated is Model B, the S-amino PBI substrate in the DT-diaphorase from NCI-H460 non-small-lung cancer.

Inspection of the substrate specificity data in Tables 1-9 reveals the following features:

1. The presence of a methyl substituent on the quinone ring substantially slows DT-diaphorase reduction. Comparison of Series 1 with 2 or Series 6 and 7 shows up to 10-fold changes in specificity when the methyl substituent is present. Steric interactions between residues W105 and F106 (FIG. 1) and the methyl substituent are very likely responsible for the lower substrate specificity of methyl-substituted quinones for DT-diaphorase. Indeed, PBI analogues bearing a 7-butyl instead of a 7-methyl were poor substrates for the enzyme. {Skibo, E. S., Gordon, S., Bess, L., Boruah, R., and Heileman, J., Studies of Pyrrolo[1,2-a]benzimidazole Quinone DT-Diaphorase Substrate Activity, Topoisomerase II Inhibition Activity, and DNA Reductive Alkylation. J. Med. Chem., 1997, 40, 1327-1339.}

2. The position of the aziridinyl substituent on the quinone ring influences substrate specificity to a lesser degree than the methyl substituent, compare 1a and 3a or 6a and 9a. In these cases, the parallel walls formed by the W105 and F106 residues can accommodate the aziridinyl substituent regardless of its position. These residues likewise accommodate the aziridinyl substituent of EO9-like indole analogues. {Phillips, R. M., Naylor, M. A., Jaffar, M., Doughty, S. W., Everett, S. A., Breen, A. G., Choudry, G. A., and Stratford, I. J., Bioreductive activation of a series of indolequinones by human DT-diaphorase: Structure-activity relationships, J. Med, Chem., 1999, 42, 4071-4080}.

3. The presence of a 3-acetoxy substituent on some of the cyclopent[b]indole systems increases substrate specificity markedly over the 3-hydroxy derivatives, compare 1a and 1b or 3a and 3b. The explanation is that the acetate carbonyl is a hydrogen bond acceptor for the N—H of His194, FIG. 1. Similarily, the 3-acetate and 3-carbamido derivatives of the PBI system can hydrogen bond with this amino acid. {Huang, X., Suleman, A., and Skibo, E. B., Rational Design of Pyrrolo[1,2-a ]benzimidazole, Based Antitumor Agents Targeting the DNA Major Groove, Bioorg. Chem., 2000, Accepted Dec. Issue} However, if a methyl substituent is present on the quinone ring, the substrate changes position in the active site and the acetate does not hydrogen bond with His 194, compare 2a and 2b or 4a and 4b.

4. The highest specificities for DT-diaphorase observed in this study were the indoles bearing an ethoxycarbonyl substituent in the 2-position, 6c and 9b. The explanation for the high substrate specificities is resonance stabilization of the anion resulting from the Michael transfer of hydride by the FADH₂ cofactor. Stabilization of this anionic intermediate by resonance would also stabilize the transition state for hydride transfer resulting in a high V_{MAX} value and accordingly a high substrate specificity. Both the crystal structure of DT-diaphorase {Faig, M., Bianchet, M. A., Talalay, P., Chen, S., Winski, S., Ross, D., and Amzel, L. M., Structures of Recombinant Human and Mouse NAD(P)H:quinone Oxidoreductases: Species Comparison and Structural Changes with Substrate Binding and Release, Proc. Natl. Acad. Sci. U.S.A., 2000, 97, 3177-82} and the reported hydride transfer mechanism to quinines {Skibo, E. B. and Lee, C. H., Facile Oxidation of Methoxide to Formaldehyde by a Heterocyclic Quinone, J. Am. Chem. Soc., 1985, 107, 4591-4593} support the Michael-type hydride transfer to the 5-position as shown in Scheme 8. In both 6c and 9b, the resulting anion (not to be confused with the radical anion arising from hydrogen atom transfer) can be resonance delocalized into the ethoxycarbonyl substituent. The 3-α-acetoxy substituent of 6d very likely shifts the substrate in the active site so that hydride transfer occurs to the 6-position rather than the 5-position. In this case, the anion resulting from Michael addition is not stabilized by resonance withdrawal by the ethoxycarbonyl substituent and its substrate specificity is substantially lower than that of 6c.

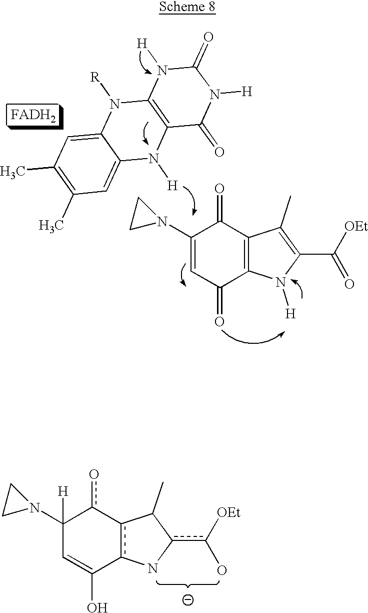

The ideal DT-diaphorase substrate appears to be an N-unsubstituted indoloquinone with an aziridinyl substituent at either the 5- or the 6-position, with a carbonyl substituent at the 2-position and a hydroxymethyl substituent in the 3-position. A following section will present evidence that designing an outstanding DT-diaphorase substrate does not necessarily translate to a good antitumor agent.

TABLE 1
DT-diaphorase kinetic parameters and DNA alkylation percentages
for Series 1-2.

| | | | | Vₘₐₓ/ | DNA |
|---|---|---|---|---|---|
| | | K_{M} | Vₘₐₓ | K_{M} | alkylation |
| COMPOUND | # | (*10⁵ M) | (* 10⁹ M/s) | (*10⁴ s⁻¹) | percentage % |
| 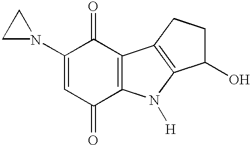 | 1a | 0.51 | 20.66 | 40.5 | 22.5 |
| 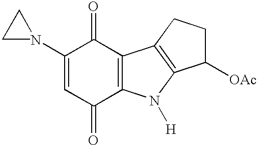 | 1b | 0.073 | 4.75 | 65.1 | 34 |
| 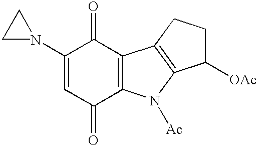 | 1c | 0.10 | 7.05 | 70.5 | 14 |
| 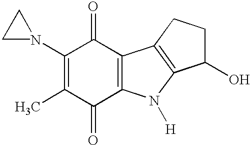 | 2a | 0.149 | 2.19 | 14.7 | 11.2 |
| 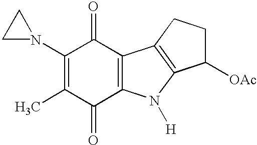 | 2b | 0.415 | 2.72 | 6.55 | 21 |
| 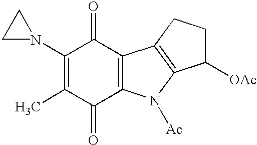 | 2c | 0.307 | 4.04 | 13.16 | 1.2 |

TABLE 2
DT-diaphorase kinetic parameters and DNA
alkylation percentages for Series 3-4.

| | | | | Vₘₐₓ/ | DNA |
|---|---|---|---|---|---|
| | | K_{M} | Vₘₐₓ | K_{M} | alkylation |
| COMPOUND | # | (*10⁵ M) | (* 10⁹ M/s) | (*10⁴ s⁻¹) | percentage % |
| 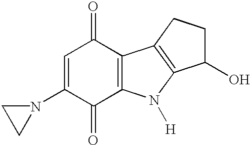 | 3a | 0.70 | 8.93 | 12.8 | 30 |
| 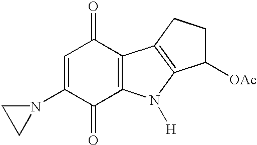 | 3b | 0.046 | 3.69 | 80.22 | 29 |
| 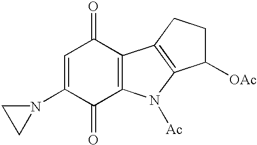 | 3c | 0.11 | 2.58 | 23.45 | 12 |
| 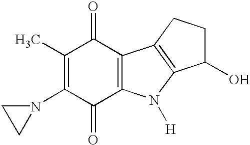 | 4a | 0.777 | 11 | 14.2 | 10 |
| 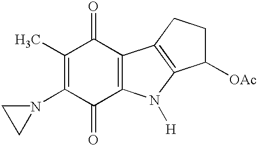 | 4b | 0.212 | 3.39 | 16 | 44 |
| 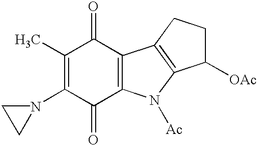 | 4c | 0.493 | 2.77 | 5.62 | 9 |

TABLE 3
DT-diaphorase kinetic parameters and DNA
alkylation percentages for Series 5-6.

| | | | | Vₘₐₓ/ | DNA |
|---|---|---|---|---|---|
| | | K_{M} | Vₘₐₓ | K_{M} | alkylation |
| COMPOUND | # | (*10⁵ M) | (* 10⁹ M/s) | (*10⁴ s⁻¹) | percentage % |
| 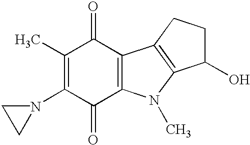 | 5a | 0.179 | 1.53 | 8.55 | 0.3 |
| 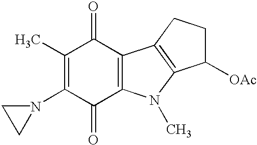 | 5b | 0.797 | 2.39 | 3.00 | 1.4 |
| 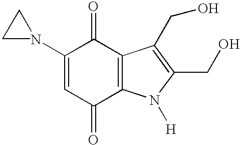 | 6a | 0.378 | 22.19 | 58.7 | 16.7 |
| 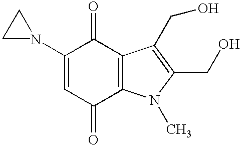 | 6b | 0.05 | 1.51 | 30.2 | 3.8 |
| 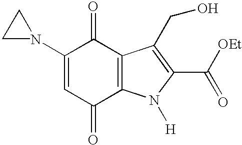 | 6c | 0.173 | 74.37 | 430 | 37 |
| 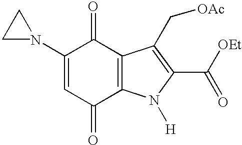 | 6d | 0.459 | 33.13 | 72.18 | 91.2 |
| 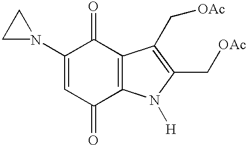 | 6e | 0.572 | 9.83 | 17.2 | 43 |

TABLE 4
DT-diaphorase kinetic parameters and DNA
alkylation percentages for Series 7-9.

| | | | | Vₘₐₓ/ | DNA |
|---|---|---|---|---|---|
| | | K_{M} | Vₘₐₓ | K_{M} | alkylation |
| COMPOUND | # | (*10⁵ M) | (* 10⁹ M/s) | (*10⁴ s⁻¹) | percentage % |
| 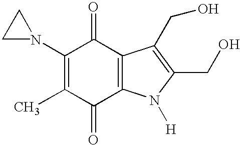 | 7a | 3.36 | 27.4 | 8.15 | 2 |
| 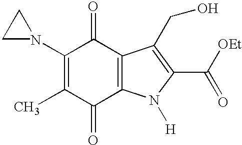 | 7b | 0.436 | 17.58 | 40.3 | 5.6 |
| 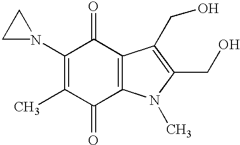 | 7c | 2.05 | 6.68 | 3.26 | 0 |
| 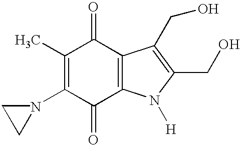 | 8a | 0.48 | 5.07 | 10.6 | 4 |
| 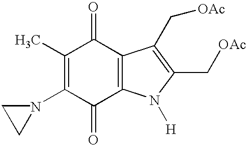 | 8b | 0.173 | 2.73 | 15.8 | 37 |
| 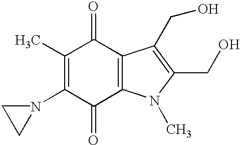 | 8c | 0.333 | 1.39 | 4.17 | 0 |
| 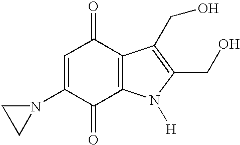 | 9a | 1.43 | 39.15 | 27.38 | 3.4 |
| 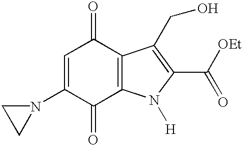 | 9b | 0.222 | 123 | 554 | 2.2 |

DNA Alkylation Screening of Series 1-9. The extent of DNA reductive alkylation was determined by catalytic reduction of the aziridinyl quinones in the presence of 600 bp calf thymus DNA. Oxidative workup afforded DNA colored blue as a result of the aminoquinone chromophore. A spectrophotometric determination of absorbance at 550 nm (ε=800 M⁻¹cm⁻¹) permitted the determination of % alkylation. {Craigo, W. A., LeSueur, B. W., and Skibo, E. B., Design of Highly Active Analogues of the Pyrrolo[1,2-a]benzimidazole Antitumor Agents, J. Med. Chem., 1999, 42, 3324-3333; Schulz, W. G., Nieman, R. A., and Skibo, E. B., Evidence for DNA Phosphate Backbone Alkylation and Cleavage by Pyrrolo[1,2-a] benzimidazoles, Small Molecules Capable of Causing Sequence Specific Phosphodiester Bond Hydrolysis, Proc. NatL Acad. Sci. U.S.A., 1995, 92, 11854-11858}. The anaerobic conditions employed for DNA reductive alkylation assays is not meant to mimic the environment of tumor cells. These conditions do permit an accurate assessment of the structural requirements for DNA alkylation by the hydroquinone species. To be sure, agents incapable of akylating DNA under these ideal conditions will not do so in the typical cellular environment. Inspection of the DNA alkylation results in Tables 1-3 revealed the following trends:

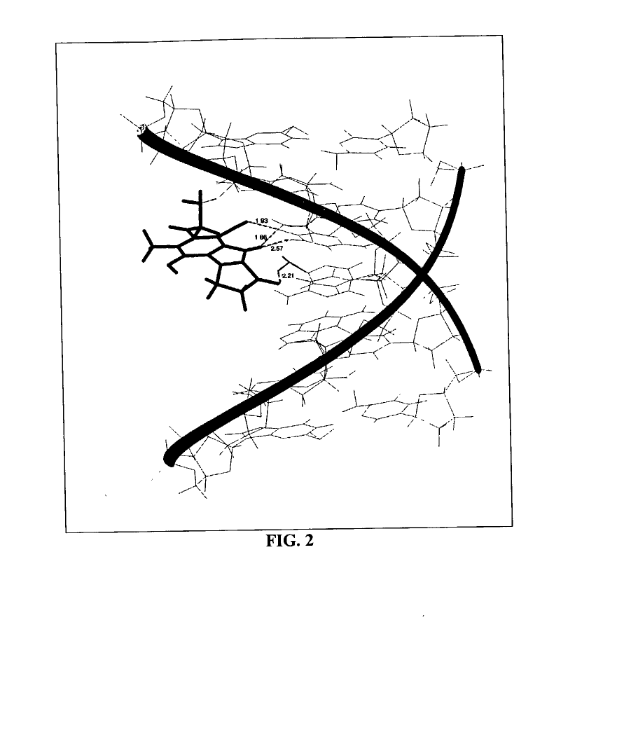

1. The presence of a 6-methyl substituent in the quinone ring of the cyclopent[b]indoles substantially reduces the % DNA alkylation compared to analogues bearing only an aziridinyl substituent (compare 1 with 2). Noteworthy exceptions are cyclopent[b]indoles 3 and 4 where a 7-methyl does not greatly influence % DNA alkylation. Similarly, the PBIs could reductively alkylate DNA with a methyl and even an n-butyl in the 7-position. {Skibo, E. S., Gordon, S., Bess, L., Boruah, R., and Heileman, J., Studies of Pyrrolo[1,2-a]benzimidazole Quinone DT-Diaphorase Substrate Activity, Topoisomerase II Inhibition Activity, and DNA Reductive Alkylation, J. Med. Chem., 1997, 40, 1327-1339} The cyclopent[b]indole DNA binding model shown in FIG. 2 rationalizes the bulk tolerance at the 7-position and the utility of this system in major groove recognition. FIG. 2 shows a Model of an N-unsubstituted cyclopent[b]indole hydroquinone in the major groove at a GC base pair with the aziridinyl group reacted with a phosphate. The 3-substituent is a hydrogen bond acceptor of the cytosine amino NH, length is 2.21 Å. The indole NH is a hydrogen bond donor to the guanine 6-carbonyl or guanine N(7), lengths are 2.57 and 1.86 Å respectively. The hydroquinone hydroxyl is a hydrogen bond donor to the guanine N(7), length is 1.86 Å.

2. N-Methylation of the cyclopent[b]indole Series 5 results in complete loss of DNA alkylation. This observation is consistent with the binding model shown in FIG. 2, where the indole NH has a hydrogen-binding role. N-Acetylation of the cyclopent[b]indole system (1c-4c) only diminishes DNA alkylation somewhat, perhaps because the carbonyl has a hydrogen-bonding role in the DNA major groove.

3. N-Methylation of the indole series (7c, 8c) also results in complete loss of DNA alkylation. It is concluded that the NH of the indole series also plays a role in DNA alkylation.

4. The presence of an acetate leaving group at the 3□-position of the indole series (6d, 6e, 8b) results in high DNA alkylation percentages. A recent publication {Skibo, E. B., Xing, C., and Groy, T., Recognition and Cleavage at the DNA Major Groove. Bioorg. Med. Chem., 2001, Accepted,} has provided evidence that elimination of the acetate affords an eneimine species capable of trapping DNA nucleophiles, Scheme 9. If an ethoxycarbonyl is present at the 2-position of the indole, the DNA alkylation percent increases with either an acetate or hydroxide leaving group at the 3□-position. The ethoxycarbonyl group lowers the pKa of the indole NH facilitating formation of the imine species, even when a weak hydroxide leaving group is present, Scheme 9.

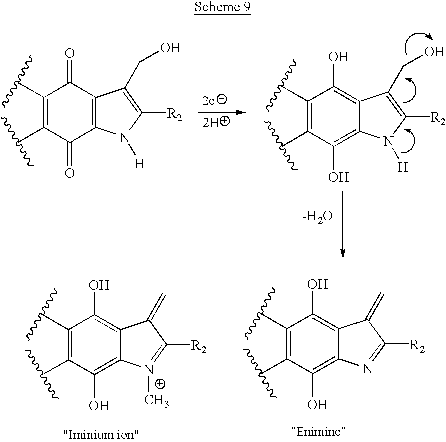

By using the guidelines above, it has been possible to design excellent DNA reductive alkylating agents with a high substrate specificity for DT-diaphorase, such as 6c and 6d.

Cytotoxic and Cytostatic Screening of Series 1-9. Provided in Table 5 are the cytostatic/cytotoxic parameters and cancer specifities for selected compounds from Series 1-9. The cytostatic parameters include GI₅₀ and TGI, which are the concentrations of drug required for 50% growth inhibition and total growth inhibition, respectively. The cytotoxic parameter is the LC₅₀, which is the concentration required for 50% cell kill. These in vitro data were obtained under the In Vitro Cell Line Screening Project at the National Cancer Institute. {Paull, D. K., Shoemaker, R. H., Hodes, L., Monks, A., Scudiero, D. A., Rubinstein, L., Plowman, J., and Boyd, M. R., Display and Analysis of Differential Activity of Drugs Against Human Tumor Cell Lines: Development of Mean Graph and COMPARE Algorithm, J. Natl. Cancer Inst., 1989, 81, 1088-1092; Boyd, M. R., Status of the NCI Preclinical Antitumor Drug Discovery Screen, Principles and Practices of Oncology (PPO updates), 1989, 3 (10),} {Paull, D. K., Shoemaker, R. H., Hodes, L., Monks, A., Scudiero, D. A., Rubinstein, L., Plowman, J., and Boyd, M. R., Display and Analysis of Differential Activity of Drugs Against Human Tumor Cell Lines: Development of Mean Graph and COMPARE Algorithm, J. Natl. Cancer Inst., 1989, 8l, 1088-1092; Boyd, M. R. Status of the NCI Preclinical Antitumor Drug Discovery Screen, Principles and Practices of Oncology (PPO updates), 1989, 3 (10),} By comparing the data in Table 5 with those in Tables 1-4, the following relationships were observed:

TABLE 5
Cytostatic (GI₅₀ and TGI) and cytotoxic (LC₅₀) parameters
and the cancer specificity for select members of Series 1-9

| | | | | Average | |
|---|---|---|---|---|---|
| | Log GI₅₀ | Log TGI | Log LC₅₀ | Log LC₅₀ | Cancer |
| # | NSC H460 | NSC H460 | NSC H460 | 60-Cell Line | Specificity |
| 1a | −8 | −6.99 | −6.53 | −5.66 ± 1.52 | Melanoma |
| | | | | | CNS |
| | | | | | Renal |
| 2a | −8 | −7.67 | −6.07 | −4.74 ± 2.11 | CNS |
| | −8 | −7.20 | −5.45 | −5.15 ± 2.14 | Melanoma |
| 3a | −8.14 | −7.24 | −5.75 | −5.59 ± 1.95 | Melanoma |
| | | | | | Renal |
| 4a | −8 | −6.97 | −4 | −4.14 ± 1.04 | Melanoma |
| | | | | −4.94 ± 2.85 | CNS |
| 4b | −6.95 | −6.17 | −5.91 | −4.67 ± 1.04 | None |
| 4c | −7.08 | −6.36 | −5.30 | −4.60 ± 1.12 | Melanoma |
| | −6.70 | −5.88 | −4.14 | −4.49 ± 1.31 | Renal |
| 5a | −5.9 | −5.12 | −4.30 | −4 | None |
| 6b | −8 | −8 | −8 | −5.35 ± 2.65 | NSC Lung |
| | −8 | −8 | −6.44 | −5.50 ± 2.5 | CNS |
| | | | | | Melanoma |
| | | | | | Renal |
| 6c | −8 | −7 | −6.20 | −5.79 ± 1.98 | CNS |
| | −8 | −6.95 | | | Melanoma |
| | | | | | Renal |
| 6d | −7.43 | −6.77 | −6.22 | −5.62 ± 1.05 | None |
| 7a | −8 | −7.49 | −5.93 | −5.54 ± 1.89 | Non-Small |
| | | | | | Cell |
| | | | | | Lung & |
| | | | | | Melanoma |
| 7b | −8 | −7.99 | −7.06 | −5.69 ± 2.31 | Non-Small |
| | −8 | −7.96 | −6.11 | −5.75 ± 2.25 | Cell |
| | | | | | Lung & |
| | | | | | Melanoma |
| 8b | −7.45 | −6.75 | −6.22 | −4.52 ± 1.7 | Non-Small |
| | −7.63 | −6.71 | −5.61 | −4.61 ± 2.5 | Cell |
| | | | | | Lung & |
| | | | | | Melanoma |
| 8c | −7.41 | −6.66 | −6.02 | −4.61 ± 1.64 | Melanoma |
| | −7.27 | −6.66 | −6.21 | −4.67 ± 2.48 | |
| 9a | −6.86 | −5.94 | −4.93 | −5.07 ± 1.43 | None |
| 9b | −5.90 | −5.55 | −5.21 | −5.20 ± 1.04 | None |
| | −5.80 | −5.39 | −4.95 | −5.32 ± 1.14 | |

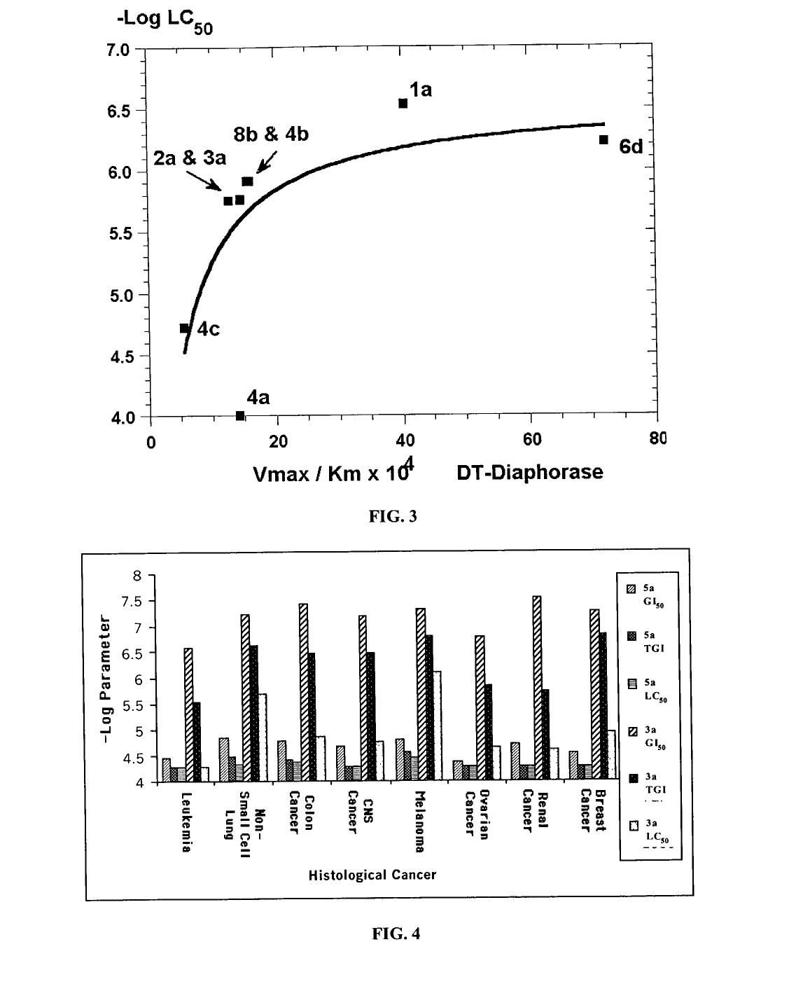

1. The data in Table 5 were used to determine the relationship between H460 DT-diaphorase substrate specificity and cytotoxicity against the H460 cell line. The −log LC₅₀ vs. V_{MAX}/K_{M} plot shown in FIG. 3 includes only DNA alkylating agents exhibiting ≧10% alkylation in our assay. FIG. 3 is a Plot of −LogLC₅₀ for Series 1-9 compounds in NCI-H460 non-small-lung cancer cell lines versus the specificity for recombinant DT-diaphorase from the same cell line. The solid line was generated from equation 1. This plot reveals that a saturating relationship exists wherein the −log LC₅₀ value of 6.6 was approached with increasingly large values for substrate specificity (V_{MAX}/K_{M} >50×10⁻⁴ s⁻¹). Compound 6c, with a V_{MAX}/K_{M} value of 430×10⁻⁴ s⁻¹ and an −log LC₅₀ value of 6.2, is at saturating cytotoxicity (not shown in FIG. 3). The solid curve in FIG. 3 was computer-generated from equation 1

−LC₅₀=A(Vₘₐₓ/Kₘ)/[B+(Vₘₐₓ/Kₘ)] (Eq1)

where −LC₅₀ is the concentration required for 50% cell kill, V_{MAX}/K_{M} is the substrate specificity for DT-diaphorase, and A and B are constants. At high substrate specificities the value of constant A approaches 6.6, the −log LC₅₀ value at saturation.

The presence of a saturating relationship suggests that reductive activation is cytotoxicity-limiting only for substrate specificities lower than 50×10⁻⁴ s⁻¹. At substrate specificities somewhat higher than 50×10⁻⁴ s⁻¹, reduction is rapid and processes such as DNA alkylation become cytotoxicity limiting. The DT-diaphorase saturating relationship has also been observed at constant antitumor agent doses, while the concentration of the enzyme was increased in the tumor cell. {Winski, S. L., Swann, E., Hargreaves, R. H. J., Butler, J., Moody C. J., and Ross, D., Relationship Between NQO1 Levels in a Series of Stably Transfected Cell Lines and Susceptibility to Antitumor Quinones, Biochem. Pharmacol., 2001, 61, 1509-1516}. Increasing the enzyme concentration no doubt results in increasingly rapid reductive activation eventually leading to constant cytotoxicity.

There have been other DT-diaphorase cytotoxicity structure-activity studies reported in the literature. {Beall, H. D., Winski, S., Swann, E., Hudnott, A. R., Cotterill, A. S., OSullivan, N., Green, S. J., Bien, R., Siegel, D., Ross, D., and Moody, C. J., Indolequinone Antitumor Agents: Correlation Between Quinone Structure, Rate of Metabolism by Recombinant Human NAD(P)H:quinone Oxidoreductase, and In Vitro Cytotoxicity, J. Med. Chem. 1998, 41, 4755-4766; Phillips, R. M., Naylor, M. A., Jaffar, M., Doughty, S. W., Everett, S. A., Breen, A. G., Choudry, G. A., and Stratford, 1. J., Bioreductive Activation of a Series of Indolequinones by Human DT-diaphorase: Structure-activity Relationships, J. Med. Chem., 1999, 42, 4071-4080}. These studies found that cytotoxicity correlated with the velocity of DT-diaphorase reduction, but without discernable saturation. Our study was carried out differently in that substrate Specificity (V_{MAX}K_{M}) rather than velocity was employed to generate the relationship. The use of velocity terms in enzyme kinetic studies is risky because such terms can include nonproductive equilibria constants, whereas such constants cancel out in the V_{MAX}K_{M} expression. {Fersht, A. R; The Hydrogen Bond in Molecular Recognition, Trends Biochem. Sci., 1987, 12, 301-304}

2. The TGI and GI₅₀ parameters are constant even with increasing DT-diaphorase specificity, except for those compounds incapable of reductively alkylating DNA. The cytostatic parameters (TGI and GT₅₀) for the NSC H460 cell line shown in Table 5 are all in the range of −log=7-8. The approximately constant cytostatic activity could be explained by the saturation phenomenon where reductive activation is not the limiting process. The limiting process would be the step following reductive activation, DNA alkylation.

3. There is another cytotoxic/cytostatic mechanism beside DNA alkylation. Compounds with low DNA alkylation capability, such as 5a and 9b, generally exhibit low cytostatic and cytotoxic activity (−log=5-6). The bar graph in FIG. 4 compares the cytostatic/cytotoxic activities of 5a with the DNA reductive alkylating agent 3a. Although both compounds possess nearly the same substrate specificity for DT-diaphorase, compound 5a is inactive against all histological cancer types while 3a exhibits significant cytostatic/cytotoxic activity. In contrast, some poor DNA alkylating agents, such as quinones 7a and 9b, possess significant cytotoxic and cytostatic properties. In addition, a −log LC₅₀ vs. V_{MAX}/K_{M} plot of only the compounds with low DNA alkylation capability revealed no clear correlation. Clearly DT-diaphorase reductive activation and DNA reductive alkylation is not required for cytotoxic and cytostatic activity. These quinones may be activated one-electron reduction and afford oxygen radicals as was observed for EO-9. {Butler, J., Spanswick, V. J., and Cummings, J., The Autoxidation of the Reduced Forms of Eo9, Free Radical Research, 1996, 25, 141-148; Butler, J. Redox cycling antitumor drugs, 1998, 131-159}FIG. 4. Bar graph of −Log of TGI, GI₅₀, and LC₅₀ for 5a and 3a versus histological cancer type. The −Log parameter values are the average of 6-8 cell lines within each histological cancer

4. High DT-diaphorase specificity and/or high DNA alkylation percentages do not result in high cancer specificity. Some members of series 1-9 possess high DT-diaphorase specificity and/or high DNA alkylation capability. Examples include 6d (91.2% of DNA bases reductively activated, 72×10⁻⁴ M⁻¹s⁻¹ DT-diaphorasc specificity), 9b (2% of DNA bases reductively activated, 554×10⁻³ M⁻¹s⁻¹ DT-diaphorase specificity), and 6c (37% of DNA bases reductively activated, 430×10⁻⁴ M⁻¹s⁻¹ DT-diaphorase specificity). The best DT-diaphorase substrate and the best DNA alkylating agent, 9b and 6d respectively, possess no specificity for the histological cancer types. Cancer specificity is measured from the ± value under the “Average Log LC₅₀ 60-Cell Line” heading of Table 5. A ± value greater than 2 indicates a greater than 100-fold higher/lower cytotoxicity in some histological cancer types compared to the mean cytotoxicity.

The DT-diaphorase levels in the National Cancer Institute 60-cell line in vitro screen have been determined and correlated with the cytotoxicity of antitumor agents activated by 2-electron reduction. {Fitzsimmons, S. A., Workman, P., Grever, M., Paull, K., Camalier, R., and Lewis, A. D., Reductase Enzyme Expression Across the National Cancer Institute Tumor Cell Line Panel: Correlation with Sensitivity To Mitomycin C and EO9, J. Natl. Cancer Inst., 1996, 88, 259-69}. Sensitive cancers usually include NSC lung and colon cancers (and to a lesser extent CNS, melanoma, and renal cancers) by virtue of their elevated DT-diaphorase levels. An excellent DT-diaphorase substrate such as 9b will be rapidly reduced regardless of the DT-diaphorase concentration in the cell resulting in broad cytotoxicity. Generally quinones with DT-diaphorase specificities below 50×10⁻⁴ M⁻¹, such as 2a, 3a, 7a and 7b possess high specificities for high DT-diaphorase cancers.

A high percentage of DNA reductive alkylation by 6d as well as 4b also results in loss of cancer specificity (± values of ˜1). These substrates are highly efficiently DNA reductive alkylating agents and function even at low DT-diaphorase concentrations, because only a low concentration is needed for DNA alkylation, resulting in the loss of cancer specificity. An exception seems to be 6c since it possesses high cancer specificity even though it is an excellent DT-diaphorase substrate and DNA reductive alkylating agent.

In vivo Screening Results

Shown in Tables 6 and 7 are B16 melanoma syngraft assays {Griswold, D. P., Consideration of the Subcutaneously Implanted B16 Melanoma as a Screening Model for Potential Anticancer Agents, Cancer Chemother Reports, 1972, 3, 315-324} of some members of Series 1-9, as well as related compounds previously reported from this laboratory. {Xing, C. G., Wu, P., Skibo, E. B., and Dorr, R. T., Design of cancer-specific antitumor agents based on aziridlinylcyclopent[b]indoloquinones, J. Med. Chem., 2000, 43, 457-466}. Each agent was evaluated at three doses in C57/bl mice: 2, 3 or 5 mg/kg/day, on days 1, 5, and 9 after subcutaneous tumor implantation of 10⁵ cells in the front flank on day 0. “Toxic” means that there was early lethality, or ≧50% lethality prior to any deaths in the control group. The treated over control values (T/C) were measured at day 25 of the study. A T/C value <40% is considered active.

The data in Table 6 reveals that cyclopent[b]indoles 3b and 3c were toxic particularly at the 5 mg/kg/day dose. In contrast, cyclopent[b]indole 4c was active at all doses while 4a possessed modest activity, all without any toxicity. The toxicity of Series 3 cyclopent[b]indoles may be due to their higher DT-diaphorase substrate specificity and DNA alkylating capability compared to the Series 4 analogues, see Table 2. However, the presence of a good leaving group in the 3-position (acetate of 4c) is required for antitumor activity. Therefore analogues possessing a poor leaving group at the 3-position, hydroxide or methoxide of 4a and 33 in Table 6, possess poor in vivo activity.

The in vivo results for the indole-based systems 6, 7, and 8 are provided in Table 7 along with the results of simpler indole analogues 34-37. {Xing, C. G., Wu, P., Skibo, E. B., and Dorr, R. T., Design of Cancer-specific Antitumor agents Based on Aziridlinylcyclopent[b]indoloquinones, J. Med. Chem., 2000, 43, 457-466}. These data clearly show that the presence of hydroxymethyl substituents, particularly in the 3-position, dramatically increases toxicity. In contrast, the simpler methylated indole analogues 33 and 34 exhibit substantial antitumor activity with toxicity only seen at the highest dose. If a hydroxymethyl is present at the 2-position of the indole, 35, antitumor activity is retained without much toxicity. The ester derivative 36 is inactive at all doses. It is only when the 3-hydroxymethyl group or its ester is present, Series 6, 7, and 9, that toxicity overwhelms antitumor activity. The simplest explanation for the above findings is that the “eneimine” alkylating agent arising from quinone reduction {Skibo, E. B., Xing, C., and Groy, T., Recognition and Cleavage at the DNA Major Groove, Bioorg. Med. Chem., 2001, Accepted,} and then loss of water from 3-hydroxymethyl derivatives, as illustrated in Scheme 9, is largely responsible for toxicity. DNA alkylation studies now underway indicate that this alkylating species reacts with the guanine N(7)- positions randomly. In contrast, the iminium ion that will arise from N-methylated indoles 6b and 8c will randomly alkylate oxygen and nitrogen centers on DNA. {Ouyang, A. and Skibo, E. B., The Iminium Ion Chemistry of Mitosene DNA Alkylating Agents. Enriched ¹³C-NMR Studies. Biochemistry 2000, 39, 5817-5830}.

TABLE 6
In vivo T/C (Treated/Control) data from the B16 melanoma
model. “NA” means Not Active. “Toxic” means ≧ 50%
lethality before any control growth deaths.

| | | 3 mg/Kg/ | |
|---|---|---|---|
| Compound | 2 mg/Kg/Day | Day | 5 mg/Kg/Day |
| 3b | NA | NA | Toxic |
| 3c | Toxic | Toxic | Toxic |
| 4a | 75% | 45% | |
| 4c | 37% | 15% | 15% |
| 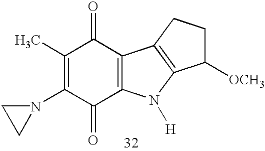 | NA | NA | NA |

TABLE 7
In vivo T/C (Treated/Control) data from the B16 melanoma
model. “NA” means Not Active. “Toxic” means ≧ 50%
lethality before any control growth deaths.

| | 2 mg/ | 3 mg/Kg/ | 5 mg/ |
|---|---|---|---|
| Compound | Kg/Day | Day | Kg/Day |
| 6a | Toxic | Toxic | Toxic |
| 6b | Toxic | Toxic | Toxic |
| 7a | 36% | Toxic | Toxic |
| 8a | 54% | Toxic | Toxic |
| 8b | 74% | Toxic | Toxic |
| 8c | NA | NA | Toxic |
| 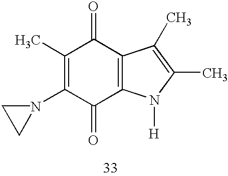 | 22% | Toxic | Toxic |
| 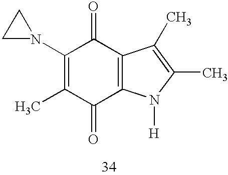 | 10% | 60% | Toxic |
| 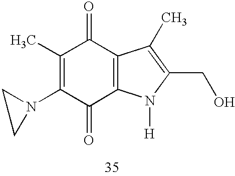 | 61% | 37% | 34% |
| 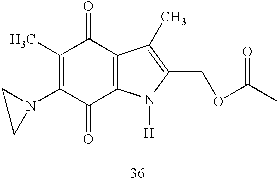 | NA | NA | NA |
| 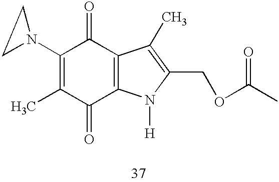 | 47% | Toxic | Toxic |

### Conclusions

A large number of aziridinyl quinones represented by Series 1-9 were studied with respect to their DT-diaphorase substrate activity, DNA reductive alkylation, cytostatic/cytotoxic activity, and in vivo activity. As a result, a number of generalizations were made that will be useful in future drug design efforts.

DT-Diaphorase Substrate Design. DT-diaphorase substrate specificity of indole- and cyclopent[b]indole-based aziridinyl quinones is highly dependent on the substituents present. Methylation of the quinone ring reduces substrate specificity while the position of the aziridinyl ring is less critical. Hydrogen bond acceptors at the 3-position cyclopent[b]indole system will also increase substrate specificity perhaps due to a hydrogen bonding interaction with the N—H of His194. The presence of an ethoxycarbonyl group at the 2-position of the indole system results in large increases in DT-diaphorase specificity perhaps due to resonance stablization of the anion arising from hydride transfer. By varying substituents in a systematic fashion, it was possible to design substrates with a 100-fold range of DT-diaphorase specificity.

DT-Diaphorase-Cytotoxicity QSAR. A comparison was made between the substrate specificity for human recombinant DT-diaphorase and the cytotoxicity of DNA alkylating and nonalkylating agents.

The DNA alkylating data were fit to a saturating relationship wherein the cytotoxicity became constant with DT-diaphorase substrate specificities (V_{MAX}/K_{M})>50×10⁻¹ s⁻¹, see Equation 1 and FIG. 3. The interpretation of this relationship is that reductive activation is no longer rate limiting at high DT-diaphorase substrate specificities. The evidence presented in this report indicates that high DT-diaphorase substrate specificity, defined as >50×10⁻⁴ s⁻¹, is not a desirable feature in the indole and cyclopent[b]indole aziridinyl quinones. High substrate specificity results in a loss of selectivity for histological cancer types with high DT-diaphorase levels as well as toxicity in vivo. The application of these conclusions to other aziridinyl quinones await study in our DT-diaphorase and DNA reductive alkylation assays.

The DNA nonalkylating agents did not show a correlation between the cytotoxicity and DT-diaphorase substrate specificities (V_{MAX}/K_{M}). These quinones are not cytotoxic as a result of two-electron reduction and may be activated one-electron reduction as was observed for EO-9. {Butler, J., Spanswick, V. J., and Cummings, J., The Autoxidation of the Reduced Forms of Eo9, Free Radical Research, 1996, 25, 141-148; Butler, J., Redox cycling antitumor drugs, 1998, 131-159}.

DNA Reductive Alkylating Agent Design. The structural features required for DNA alkylating agent design. The presence of a 6-methyl in the cyclopent[b]indoles substantially reduces DNA reductive alkylation for steric reasons. Likewise, an N-methyl in either the indole or the cyclopent[b]indole systems hinders DNA reductive alkylation resulting in near zero percent alkylation in some cases. Perhaps the most important prerequisite for efficient DNA reductive alkylation by the indole system is a leaving group in the 3α-position, either a hydroxide or acetate. When the indole system is N-unsubstituted, leaving group elimination affords the highly reactive eneimine functional group. Ongoing studies have revealed that indole systems able to form the eneimine upon reductive activation are efficient DNA crosslinkers. The members of Series 1-9 possess a wide range of DNA reductive alkylation capability, from near 0% to overalkylation at ˜90%. While some DNA alkylation was required for cytostatic and cytotoxic activity, too much alkylation results in loss of cancer selectivity as well as increased in vivo toxicity. Indeed, the most lethal compounds are the indole systems with an acetate or hydroxyl leaving group at the 3α-position. We conclude that poor DNA alkylating agents (according to our assay) show the lowest toxicity with the highest antitumor activity. For these compounds, redox recycling with oxygen radical generation {Butler, J., Redox Cycling Antitumor Drugs, 1998, 131-159} may be responsible for cytotoxicity.

### Experimental Section

All solutions and buffers for kinetic, DNA, and electrophoresis studies used doubly distilled water. All analytically pure compounds were dried under high vacuum in a drying pistol over refluxing toluene. Elemental analyses were run at Atlantic Microlab, Inc., Norcross, Ga. All TLCs were performed on silica gel plates using a variety of solvents and a fluorescent indicator for visualization. IR spectra were taken as thin films and the strongest absorbances reported. ¹H NMR spectra were obtained from a 300 MHz spectrometer. All chemical shifts are reported relative to TMS.

The synthesis of new compounds are outlined below in the order found in Schemes 1-6.

Substituted 1,4-dihydrocyclopent[b]indol-3(2H)-ones (10) were prepared as previously described for 10b.

6-Methoxy-1,4-dihydrocyclopent[b]indol-3(2H)-one(10a). Yield20%; mp 190° C.; TLC (dichloromethane:MeOH 95:5), R_{f}=0.75; ¹HNMR(CDCl₃) δ9.11 (1H, bs, indole proton), 7.57(1H, d, J=9.0 Hz, 8-proton), 6.89(1H, d, J=2.4 Hz, 5-proton), 6.83(1H, dd, J=2.4 Hz, J=9.0 Hz, 7-proton), 3.89(3H, s, 6-methoxy), 3.09 and 3.00(4H, m, methylenes of cyclopentyl); IR(KBr pellet)3450, 3176, 3090, 3026, 2957, 2924, 2843, 1662, 1620, 1535, 1257, 1197, 1136, 1047, 912, 821 cm⁻¹; MS(EI)201(M⁺), 186(M⁺-CH₃), 173, 158, 130. Anal. Calcd(C₁₂H₁₁NO₂)C, H, N.

7-Methoxy-1,4-dihydrocyclopent[b]indol-3(2H)-one(10c).Yield40%. mp 252° C.; TLC, (dichloromethane:MeOH 95:5), R_{f}=0.70; ¹HNMR(CDCl₃) δ8.42(1H, bs, indole proton), 7.36(1H, dd, J=1.5 Hz, J=9.0 Hz, 6-proton), 7.09(1H, d, J=1.5 Hz, 8-proton), 7.06(1H, d, J=9.0 Hz, 5-proton), 3.87(3H, s, 7-methoxy), 3.09 and 3.00(4H, m, methylenes of cyclopentyl); IR(KBr pellet)3435, 3159, 2997, 2926, 2854, 1589, 1651, 1626, 1537, 1491, 1450, 1404, 1309, 1276, 1217, 1099, 1045 cm⁻¹;MS(EI) 201(M⁺), 186(M⁺-CH₃), 173, 158, 130. Anal. Calcd(C₁₂H₁₁NO₂)C, H, N.

7-Methoxy-6-methyl-1,4-dihydrocyclopent[b]indol-3(2H)-one(10d).Yield 23%; mp 222° C.; TLC, (dichloromethane:MeOH 95:5), R_{f}=0.80; ¹HNMR(CDCl₃) δ8.30(1H, bs, indole proton), 7.19 and 6.99(2H, 2s, 5,8-protons), 3.89(3H, s, 7-methoxy), 3.09 and 3.00(4H, m, methylenes of cyclopentyl), 2.35(3H, s, 6-methyl); IR(KBr pellet)3418, 3176, 2920, 2836, 1666, 1556, 1487, 1446, 1307, 1207, 1095, 1047, 815 cm⁻¹. MS(EI)215(M⁺), 200(M⁺-CH₃), 187, 172, 115. Anal. Calcd(C₁₃H₁₃NO₂)C, H, N.

Nitro-1,4-dihydrocyclopent[b]indol-3(2H)-ones (11). To a solution of 10 mmol 10 in 200 mL of conc H₂SO₄, cooled at −20° C., was added a solution of 10.5 mmol KNO₃ in 20 mL of conc H₂SO₄. The solution was stirred at that temperature for 40 min and was poured over 500 g ice, the solution was extracted 5× with 200 mL portions of CH₂Cl₂. The extracts were washed with NaHCO₃, dried over Na₂SO₄, and vacuum dried. The product was crystallized from CH₂Cl₂ and hexane.

6-Methoxy-5-nitro-1,4-dihydrocyclopent[b]indol-3(2H)-one (11a). Yield 32%; Mp 228° C.; TLC, (dichloromethane:MeOH 95:5), R_{f}=0.68; ¹HNMR(CDCl₃) δ10.01(1H, bs, indole proton), 7.94 and 7.00(2H, 2d, J=8.7 Hz, 7,8-protons), 4.11(3H, s, 6-methoxy), 3.12 and 3.04(4H, m, methylenes of cyclopentyl); IR(KBr pellet) 3460, 3375, 3269 2926, 2850, 1672, 1626, 1550, 1514, 1330, 1246, 1182, 1118, 1006, 962, 812 cm⁻¹; MS(EI)246(M⁺), 231(M⁺-CH₃), 218, 199, 186, 170, 158, 142. Anal. Calcd(C₁₂H₁₀N₂O₄)C, H, N.

7-Methoxy-8-nitro-1,4-dihydrocyclopent[b]indol-3(2H)-one (11c). Yield65%; Mp 293° C.; TLC, (dichloromethane:MeOH 95:5), R_{f}=0.60; ¹HNMR(CDCl₃) δ9.49(1H, bs, indole proton), 7.69 and 7.23(2H, 2d, J=8.7 Hz, 5,6,-protons), 4.02(3H, s, 7-methoxy), 3.30 and 3.00(4H, m, methylenes of cyclopentyl); IR(KBr pellet)3437, 3192, 2928, 2854, 2660, 1672, 1628, 1568, 1518, 1329, 1271, 1244, 1182, 1091, 1049, 1001, 815 cm⁻¹; MS(EI)246(M⁺), 229(M⁺-OH), 216, 198, 170. Anal. Calcd(C₁₂H₁₀N₂O₄)C, H, N.

7-Methoxy-6-methyl-8-nitro-1,4-dihydrocyclopent[b]indol-3(2H)-one (11d). Yield 55%; mp 240° C.; TLC(dichloromethane:MeOH 90:10), R_{f}=0.85; ¹HNMR(CDCl₃) δ9.45(1H, bs, indole proton), 7.55(H, s, 5-protons), 3.95(3H, s, 7-methoxy), 3.30 and 3.00(4H, m, methylenes of cyclopentyl), 2.49(3H, s, 6-methyl); IR(KBr pellet)3448, 3203, 3059, 2960, 2926, 2856, 2652, 1672, 1489, 1352, 1276, 1230, 1093, 1022, 972, 864, 823 cm⁻¹; MS(EI)260(M⁺), 243(M⁺-OH), 230, 213, 185, 156, 143, 128. Anal. Calcd(C₁₃H₁₂N₂O₄)C, H, N.

3-Hydroxy-5 or 8-nitro-1,2,3,4-Tetrahydrocyclopent[b]indoles (12). To a solution of 5 mmol of 11 in 100 mL of MeOH was added 400 mg of NaBH₄ and the solution was stirred at room temperature for 20 min. The solution was then mixed with 400 mL of H₂O and extracted 4× with 100 mL of CH₂Cl₂. The combined extracts were dried over Na₂SO₄ and vacuum dried. The product was crystallized from CH₂Cl₂ and hexane.

3-Hydroxy-6-methoxy-5-nitro-1,2,3,4-tetrahydrocyclopent[b]indole (12a). Yield 94%; mp 192° C.; TLC (dichloromethane:MeOH 95:5), R_{f}=0.45; ¹HNMR(CDCl₃) δ9.80(1H, bs, indole proton), 7.68 and 6.86(2H, 2d, J=8.7 Hz, 7,8-protons), 5.36(1H, m, 3-hydroxymethylene proton), 4.05(3H, s, 6-methoxy), 3.02, 2.75 and 2.38(4H, m, methylenes of cyclopentyl), 1.80(1H, d, J=7.2 Hz, 3-hydroxymethylene hydroxy); IR(KBr pellet)3435, 2928, 2852, 1626, 1566, 1508, 1334, 1290, 1184, 1112, 1057, 947, 804 cm⁻¹;MS(EI)248(M⁺), 231(M⁺-OH), 215, 200, 184, 172, 154, 144. Anal. Calcd(C₁₂H₁₂N₂O₄)C, H, N.

3-Hydroxy-7-methoxy-8-nitro-1,2,3,4-tetrahydrocyclopent[b]indole (12c). Yield 85%; mp 230° C.; TLC(dichloromethane:MeOH 95:5), R_{f}=0.25; ¹HNMR(CDCl₃) δ8.19(1H, bs, indole proton), 7.45 and 6.93 (2H, 2d, J=8.7 Hz, 5,6-protons), 5.32(1H, m, 3-hydroxymethylene proton), 3.97(3H, s, 7-methoxy), 3.00, 2.82 and 2.33(4H, m, methylenes of cyclopentyl); IR(KBr pellet)3551, 3464, 3416, 3238, 3063, 2972, 2870, 1635, 1572, 1506, 1313, 1276, 1240, 1182, 1082, 954, 787 cm⁻¹; MS(EI) 248(M⁺), 231(M⁺-OH), 213, 200, 172, 154, 143. Anal. Calcd(C₁₂H₁₂N₂O₄)C, H, N.

3-Hydroxy-7-methoxy-6-methyl-8-nitro-1,2,3,4-tetrahydrocyclopent[b]indole (12d). Yield 92%; mp 185° C.; TLC (dichloromethane:MeOH 90:10), R_{f}=0.65; ¹HNMR(CDCl₃) δ8.16(1H, bs, indole proton), 7.33(1H, s, 4-protons), 5.30(1H, m, 3-hydroxymethylene proton), 3.91(3H, s, 7-methoxy), 3.00, 2.82 and 2.33(4H, m, methylenes of cyclopentyl), 2.43(3H, s, 6-methyl); IR(KBr pellet)3408, 3175, 3047, 2943, 2856, 1637, 1570, 1516, 1427, 1329, 1288, 1219, 1149, 1076, 1041, 976, 866 cm⁻¹; MS(EI)262(M⁺), 244(M⁺-H₂O), 227, 215, 197, 186, 168, 154, 143, 127, 115. Anal. Calcd(C₁₃H₁₄N₂O₄)C, H, N.

3-Hydroxy-1,2,3,4-tetrahydrocyclopent[b]indole-5,8-dione(13) A solution of 0.5 mmol of the product above in 10 mL of MeOH and 40 mL of H₂O with 150 mg 5% Pd on carbon was reduced under 50 psi of H₂ for 30 min and the catalyst was filtered off employing Celite. The filtrate was mixed with a solution of 400 mg KH₂PO₄ and 800 mg Fremy salt in 150 ml H₂O, stirred at room temperature for 3 h. The solution was extracted with 5× with 50 ml portions of CH₂Cl₂ to remove the quinone product. The extracts were dried over Na₂SO₄ and vacuum dried. The solid was purified with a flash chromatography using 10% acetone in CH₂Cl₂ as the eluent. The product was recrystallized from CH₂Cl₂ and hexane.

3-Hydroxy-6-methoxy-1,2,3,4-tetrahydrocyclopent[b]indole -5,8-dione(13a). Yield 15%; mp 167° C.; TLC (dichloromethane:MeOH 90:10), R_{f}=0.70; ¹HNMR(d₆-DMSO) δ12.34(1H, bs, indole proton), 5.72(1H, s, 5-proton), 5.27(1H, m, 3-hydroxymethylene proton), 3.74(3H, s, 6-methoxy), 2.77, 2.46 and 2.15(4H, m, methylenes of cyclopentyl); IR(KBr pellet)3493, 3136, 2989,2939,2858, 1664, 1628, 1591, 1543, 1402, 1323, 1238, 1120, 1055, 935, 848 cm⁻¹; MS(EI)233(M⁺), 215(M⁺-H₂O), 202, 190, 174, 158. Anal. Calcd(C₁₂H₁₁NO₄)C, H, N.

3-Hydroxy-7-methoxy-1,2,3,4-tetrahydrocyclopent[b]indole -5,8-dione(13c). Yield 18%; mp 200° C.; TLC(dichloromethane:MeOH 95:5), R_{f}=0.20; ¹HNMR(CDCl₃) δ9.12(1H, bs, indole proton), 5.69(1H, 6-protons), 5.20(1H, m, 3-hydroxymethylene proton), 3.83(3H, s, 7-methoxy), 3.01, 2.85and 2.29(4H, m, methylenes of cyclopentyl); IR(KBr pellet)3431, 3205, 3109, 3067, 2937, 2868, 1678, 1645, 1591, 1483, 1458, 1408, 1342, 1292, 1248, 1211, 1180, 1082, 1030, 949, 868, 842 cm⁻¹; MS(EI)233(M⁺), 215(M⁺-H₂O), 200, 190, 187, 176, 172, 162, 158, 144,130. Anal. Calcd(C₁₂H₁₁NO₄)C, H, N.

3-Hydroxy-7-methoxy-6-methyl-1,2,3,4-tetrahydrocyclopent[b]indole -5,8-dione(13d). Yield 13%; mp 215° C.; TLC (dichloromethane:MeOH 90:10), R_{f}=0.60; ¹HNMR(CDCl₃) δ9.10(1H, bs, indole proton), 5.18(1H, m, 3-hydroxymethylene proton), 4.02(3H, s, 7-methoxy), 3.00, 2.78 and 2.32(4H, m, methylenes of cyclopentyl), 1.97(3H, s, 6-methyl); IR(KBr pellet)3435, 3207, 3013, 2926, 2852, 1637, 1498, 1450, 1408, 1373, 1317, 1290, 1217, 1168, 1105, 1039, 985, 949, 808 cm⁻¹; MS(EMI)247(M+), 229 (M⁺-H₂O), 214, 204, 201, 190, 186, 176, 172, 158, 144. Anal. Calcd(C₁₃H₁₃NO₄)C, H, N.

Aziridinyl-3-hydroxy-1,2,3,4-tetrahydrocyclopent[b]indole -5,8-dione(Series a) To a solution of 0.3 mmol of 13 in 30 mL of MeOH was added 0.5 mL of ethylenimine and the solution was stirred at RT for 8 h. The solvent was vacuum removed and the residue was applied to a flash chromatography using 20% acetone in CH₂Cl₂ as eluent. The product was crystallized from CH₂Cl₂ and hexane.

7-Aziridinyl-3-hydroxy-1,2,3,4-tetrahydrocyclopent[b]indole-5,8-dione(1a) Yield 93%; mp 145° C.; TLC (dichloromethane:MeOH 90:10), R_{f}=0.55; ¹HNMR(CDCl₃) δ9.26(1H, bs, indole proton), 5.81(1H, 6-proton), 5.20(1H, m, 3-hydroxymethylene proton), 2.96, 2.74and 2.33(4H, m, methylenes of cyclopentyl), 2.22(4H, s, 7-aziridinyl); IR(KBr pellet)3418, 3283, 2946, 2870, 1626, 1578, 1476, 1364, 1254, 1156, 1078, 946, 846, 808 cm⁻¹; MS(EI)244(M⁺), 226(M⁺-H₂O), 215, 200, 192, 176, 137. Anal. Calcd(C₁₃H₁₂N₂O₃)C, H, N.

7-Aziridinyl-3-hydroxy-6-methyl-1,2,3,4-tetrahydrocyclopent[b]indole -5,8-dione(2a). Yield 95%; mp 220° C.; TLC (dichloromethane:MeOH 90:10), R_{f}=0.55; ¹HNMR(CDCl₃) δ9.23(1H, bs, indole proton), 5.18(1H, m, 3-hydroxymethylene proton), 2.96, 2.72and 2.36(4H, m, methylenes of cyclopentyl), 2.32(4H, s, 7-aziridinyl), 2.53(3H, s, 6-methyl); IR(KBr pellet)3420, 3192, 2928, 2856, 1664, 1629, 1560, 1498, 1458, 1377, 1292, 1230, 1074, 1039, 981, 856 cm⁻¹; MS(EI)258(M⁺), 240(M⁺-H₂O), 229, 214, 204, 190, 151. Anal. Cal.: C, 65.10; H, 5.46; N, 10.85. Found: C, 65.16; H, 5.44; N, 10.89. Anal. Calcd(C₁₄H₁₄N₂O₃)C, H, N.

6-Aziridinyl-3-hydroxy-1,2,3,4-tetrahydrocyclopent[b]indole -5,8-dione(3a) Yield 89%; mp 180° C.; TLC(dichloromethane:MeOH 90:10), R_{f}=0.50; ¹HNMR(d₆-DMSO) δ12.12(1H, bs, indole proton), 5.73(1H, s, 7-protons), 5.20(1H, m, 3-hydroxymethylene proton), 2.70, 2.46 and 2.11(4H, m, methylenes of cyclopentyl), 2.12(4H, s, 6-aziridinyl); IR(KBr pellet)3447, 3198, 3003, 2930, 2864, 1666, 1614, 1577, 1475, 1406, 1294, 1238, 1112, 1041, 949, 852, 806 cm⁻¹; MS(EI)244(M⁺), 226(M⁺-H₂O), 217, 199, 189, 171. Anal. Calcd(C₁₃H₁₂N₂O₃)C, H, N.

3-Acetoxy-6 or 7-aziridinyl-1,2,3,4-tetrahydrocyclopent[b]indole -5,8-dione(b) and 6 or 7-Aziridinyl-3-acetoxy-4-acetyl-1,2,3,4-tetrahydrocyclopent[b]indole -5,8-dione(Series c) To a solution of 0.4 mmol of 1-3a in 10 ml 10% acetone in CH₂Cl₂ with 50 mg DMAP present was added 3×60λ of acetic anhydride in every 3 min. The solution was then directly applied to flash chromatography using 10% acetone in CH₂Cl₂ as the eluent. The solution was washed with saturated NaHCO₃, dried over Na₂SO₄, and vacuum dried. The product was precipitated from CH₂Cl₂ and hexane.

3-Acetoxy-7-aziridinyl-1,2,3,4-tetrahydrocyclopent[b]indole -5,8-dione(1b) Yield 42%; mp 165° C.; TLC (dichloromethane:MeOH 90:10), R_{f}=0.65; ¹HNMR(CDCl₃) δ9.20(1H, bs, indole proton), 5.83(1H, 6-protons), 5.66(1H, m, 3-hydroxymethylene proton), 2.99, 2.76and 2.21(4H, m, methylenes of cyclopentyl), 2.21(4H, s, 7-aziridinyl), 2.06(3H, s, 3-acetyl methyl); IR(KBr pellet)3448, 3225, 3065, 2926, 2872, 1720, 1641, 1585, 1510, 1475, 1367, 1288, 1251, 1163, 1018, 985, 812 cm⁻¹; MS(EI)286(M⁺), 258(M⁺-CO), 243, 226, 211, 199, 170, 142, 118. Anal. Calcd(C₁₅H₁₄N₂O₄)C, H, N.

3-Acetoxy-7-aziridinyl-6-methyl-1,2,3,4-tetrahydrocyclopent[b]indole -5,8-dione(2b) Yield 45%; mp 165° C.; TLC(dichloromethante:MeOH 90:10), R_{f}=0.75; ¹H NMR(CDCl₃) δ9.13(1H, bs, indole proton), 5.66(1H, m, 3-hydroxymethylene proton), 2.99, 2.59and 2.30(4H, m, methylenes of cyclopentyl), 2.30(4H, s, 7-aziridinyl), 2.06(6H, 2s, 3-aceetyl methyl and 6-methyl); IR(KBr pellet)3454, 3311, 3043, 2926, 2843, 1732, 1663, 1572, 1473, 1382, 1239, 1009, 964, 800 cm⁻¹; MS(EI)300(M⁺), 272(M⁺-CO), 257, 240, 225, 213, 184, 156, 132. Anal. Calcd(C₁₆H₁₆N₂O₄)C, H, N.

3-Acetoxy-6-aziridinyl-1,2,3,4-tetrahydrocyclopent[b]indole-5,8-dione(3b) Yield 32%; mp 155° C.; TLC(dichloromethane:acetone 80:20), R_{f}=0.45; ¹HNMR(CDCl₃) δ9.31(1H, bs, indole proton), 5.92(1H, 7-protons), 5.64(1H, m, 3-hydroxymethylene proton), 3.00, 2.56 and 2.28(4H, m, methylenes of cyclopentyl), 2.24(4H, s, 6-aziridinyl), 2.05(3H, s, 3-acetyl methyl) IR(KBr pellet)3437, 3219, 3103, 1995, 1945, 2864, 1724, 1643, 1583, 1481, 1369, 1296, 1236, 1112, 1018, 991, 956, 891, 850 cm⁻¹; MS(EI)286(M⁺), 258(M⁺-CO), 244, 226, 215, 199, 170. Anal. Calcd(C₁₅H₁₄N₂O₄)C, H, N.

7-Aziridinyl-3-acetoxy-4-acetyl-1,2,3,4-tetrahydrocyclopent[b]indole -5,8-dione(1c) Yield 45%; mp 140° C.; TLC (dichloromethane:acetone 80:20), R_{f}=0.65; ¹HNMR(CDCl₃) δ6.20(1H, m, 3-hydroxymethylene proton), 5.90(1H, s, 6-proton), 2.99, 2.41and 2.20(4H, m, methylenes of cyclopentyl), 2.74(3H, s, 4-acetyl methyl), 2.22(4H, s, 7-aziridinyl), 2.06(3H, s 3-acetyl methyl); IR(KBr pellet)3431, 3005, 2928, 2870, 1734, 1672, 1639, 1593, 1491, 1371, 1246, 1186, 1157, 1095, 1020, 856 cm⁻¹; MS(EI)328(M⁺), 300(M⁺-CO), 286, 244, 226, 199, 171, 132. Anal. Calcd(C₁₇H₁₆N₂O₅)C, H, N.

7-Aziridinyl-3-acetoxy-4-acetyl-6-methyl-1,2,3,4-tetrahydrocyclopent[b]indole -5,8-dione(2c) Yield 48%; mp 156° C.; TLC (dichloromethane:acetone 80:20), R_{f}=0.70; ¹HNMR(CDCl₃) δ6.26(1H, m, 3-hydroxymethylene proton), 2.97, 2.43and 2.22(4H, m, methylenes of cyclopentyl), 2.72(3H, s, 4-acetyl), 2.25(4H, s, 7-aziridinyl), 2.08 and 2.05(6H, 2s, 6-methyl and 3-acetyl methyl); IR(KBr pellet) 3433, 3011, 2945, 2854, 1734, 1666, 1637, 1593, 1496, 1381, 1350, 1240, 1217, 1157, 1022, 983, 933 cm⁻¹; MS(EI) 342(M⁺), 300(M⁺-acetyl), 283, 258, 240, 225. Anal. Calcd(C₁₈H₁₈N₂O₅)C, H, N. 6-Aziridinyl-3-acetoxy-4-acetyl-1,2,3,4-tetrahydrocyclopent[b]indole -5,8-dione(3c) Yield 56%; MP 167° C.; TLC(dichloromethane:acetone 80:20), R_{f}=0.65; ¹HNMR(CDCl₃) δ5.97(1H, m, 3-hydroxymethylene proton), 5.91(1H, s, 7-proton), 2.97, 2.43and 2.21(4H, m, methylenes of cyclopentyl), 2.77(3H, s, 4-acetyl methyl), 2.25(4H, s, 6-aziridinyl), 2.04(3H, s 3-acetyl methyl); IR(KBr pellet)3448, 3007, 2941, 2862, 1736, 1668, 1637, 1585, 1473, 1361, 1251, 1230, 1112, 1033, 981, 893 cm⁻¹; MS(EI)328(M⁺), 300(M⁺-CO), 286, 244, 226, 199, 171, 132. Anal. Cal.: C, 62.19; H, 4.91; N, 8.53. Found: C, 62.16; H, 4.88; N, 8.50. Anal. Calcd(C₁₇H₁₆N₂O₅)C, H, N.

4,7-Dimethyl-8-nitro-1,4-dihydrocyclopent[b]indol-3(2H)-one (14) To 0.2 mmol of 11b and 400 mg KOH in 20 mL of acetone with was added 1.0 mL of MeI followed by stirring at RT for 1 h. The reaction mixture was then neutralized with conc. HCl and extracted 4× with 30 mL portions of CH₂Cl₂. The combined extracts were dried (Na₂SO₄), concentrated to a residue, and chromographed using CH₂Cl₂ as eluent. The product was crystallized from methylene chloride using hexane. Yield 92%; mp 207° C.; TLC (dichloromethane), R_{f}−0.45; ¹HNMR(CDCl₃) δ7.49 and 7.28(2H, 2d, J=8.7Hz, 5,6-protons), 3.96(3H, s, 4-methyl), 3.14 and 2.97(4H, m, methylenes of cyclopentyl), 2.62(3H, s, 5-methyl); IR(KBr pellet)3437, 3063, 2939, 2849, 1683, 1521, 14581373, 1338, 1265, 1201, 1047, 833 cm⁻¹; MS(EI) 244(M⁺), 227(M⁺-OH), 197, 184, 169, 154. Anal. Calcd(C₁₃H₁₂N₂O₃)C, H, N.

4,7-Dimethyl-3-hydroxy-8-nitro-1,2,3,4-tetrahydrocyclopent[b]indole (15). The borohydride reduction of 14 to afford 15 was carried out as described above for the preparation of 12: Yield 90%; mp 171° C.; TLC (dichloromethane:MeOH 95:5), R_{f}=0.80; ¹HNMR(CDCl₃) δ7.37 and 7.05(2H, 2d, J=8.4 Hz, 5,6-protons), 5.36(1H, m, 3-hydroxymethylene proton), 3.82(3H, s, 4-methyl), 3.09, 2.88 and 2.33(4H, m, methylenes of cyclopentyl), 2.39(3H, s, 7-methyl), 1.65(1H, d, J=9.0 Hz, 3-hydroxymethylene hydroxy); IR(KBr pellet)3435, 3302, 3202, 2962, 2930, 1637, 1560, 1516, 1460, 1321, 1269, 117, 1045, 952, 908, 808 cm⁻¹; MS(EI) 246(M⁺), 229(M⁺-OH), 212, 199, 182, 167, 156. Anal. Calcd(C₁₃H₁₄N₂O₃)C, H, N.

4,7-Dimethyl-3-hydroxy-1,2,3,4-tetrahydrocyclopent[b]indole -5,8-dione(16) The catalytic reduction and Fremy oxidation of 15 to afford 16 was carried out as described for the preparation of 13: Yield 14%; mp 160° C.; TLC (dichloromethane:MeOH 96: 4), R_{f}=0.35; ¹HNMR(CDCl₃) δ6.35(1H, q, J=1.5 Hz, 6-proton), 5.22(1H, m, 3-hydroxymethylene proton), 3.95(3H, s, 4-methyl), 3.02, 2.73 and 2.34(4H, m, methylenes of cyclopentyl), 2.04(3H, d, J=1.5 Hz, 7-methyl), 1.72(1H, d, J=8.4 Hz, 3-hydroxymethylene hydroxy); IR(KBr pellet) 3469, 2943, 2872, 1639, 1604, 1475, 1384, 1221, 1159, 1039, 947, 877 cm⁻¹; MS(EI)231(M⁺), 214(M⁺-OH), 203, 188, 174, 160. Anal. Calcd(C₁₃H₁₃NO₃)C, H, N.

6-Aziridinyl-4,7-dimethyl-3-Hydroxy-1,2,3,4-tetrahydrocyclopent[b]indole -5,8-dione(5a) was prepared by the procedure described under Series a :Yield 32%; mp 187° C.; TLC (dichloromethane:MeOH), R_{f}=0.60; ¹HNMR(d₆-DMSO) 65.41(1H, d, J=6.6 Hz, 3-hydroxyl), 5.04(1H, m, 3-hydroxymethylene proton), 3.82(3H, s, 4-methyl), 2.65, 2.50, and 2.10(4H, m methylenes of cyclopentyl), 2.17(4H, s, 6-aziridine); IR(KBr pellet)3431, 2995, 2949, 2922, 2858, 1653, 1620, 1479, 1377, 1340, 1153, 1051, 983 cm⁻¹; MS(EI)272(M⁺), 255(M⁺-OH), 243, 227, 217. Anal. Calcd(C₁₅H₁₆N₂O₃)C, H, N.

3-Acetyl-6-aziridinyl-4,7-dimethyl-1,2,3,4-tetrahydrocyclopent[b]indole -5,8-dione(5b) Yield 50%; mp 157° C.; TLC (dichloromethane:acetone 96:4), R_{f}=0.62; ¹HNMR(CDCl₃) δ6.10(1H, m, 3-hydroxymethylene proton), 3.89(3H, s, 4-methyl), 2.96, 2.79 and 2.40(4H, m, methylenes of cyclopentyl), 2.30(4H, s, 6-aziridinyl), 2.09 and 2.08(6H, 2methyl and 3-acetyl methyl). IR(KBr pellet)3447, 3020, 2922, 2854, 1734, 1653, 1626, 1583, 1375, 1336, 1232, 1151, 1030, 856 cm⁻¹; MS(EI)314(M⁺), 299(M⁺-CH₃), 272, 255, 239, 227, 215. Anal. Calcd(C₁₇H₁₈N₂O₄)C, H, N.

2-Ethoxycarbonyllindoles (17) The synthesis of 17a, 17c was reported in literature {Allen, M. S., Hamaker, L. K., Laloggia, A. J., and Cook, J. M., Entry Into 6-Methoxy-D(+)-Tryptophans—Stereospecific Synthesis of 1-Benzenesulfonyl-6-Methoxy-D(+)-Tryptophan Ethyl- EsterSynth. Commun., 1992, 22, 2077-2102} and 17b was synthesized following the procedure described for 16a.

2-Ethoxycarbonyl-5-methoxy-6-methylindole (17b) Yield 63%; mp 165° C.; TLC (CHCl₃), R_{f}=0.55; ¹HNMR(CDCl₃) δ8.65(1H, bs, indole proton), 7.26, 7.11, and 6.99(3H, 3s, 3, 4,7-proton), 4.38(2H, q, J=7.2 Hz, methylene of 2-ethoxyl), 3.87(3H, s, 5-methoxyl), 2.33(3H, s, 6-methyl), 1.39(3H, t, J=7.2 Hz, methyl of 2-ethoxyl); IR(KBr pellet)3445, 3325, 2982, 2941, 2839, 1682, 1520, 1246, 1213, 1168, 1136, 1026, 864, 833 cm⁻¹; MS(EI)233(M⁺),218, 204, 187, 172, 144. Anal. Calcd(C₁₃H₁₅NO₃)C, H, N.

2-Ethoxycarbonyl-3-formylindoles (18) A solution of 6 mL of distilled DMF with 2 mL of distilled POC₃ in 40 mL of dried CH₂Cl₂ was stirred at 0° C. for 30 min. To this solution was slowly added 50 mmol of 18 in 30 mL of dried CH₂Cl₂ and the reaction mixture stirred at 0° C. for 30 min. To the completed reaction was added NaHCO₃ saturated solution until neutral. The product was removed from the neutralized reaction by extracting 5× with 100 mL portions of methylene chloride. The product was crystallized from dichloromethane and hexane.

2-Ethoxycarbonyl-3-formyl-5-methoxyindole (18a) Yield 87%; mp 231° C.; TLC(CHCl₃), R_{f}=0.30; ¹HNMR(CDCl₃) δ10.74(1H, s, 3-formyl), 9.20(1H, bs, indole proton), 7.90(1H,d, J=2.7 Hz, 4-proton), 7.35(1H, d, J=8.7 Hz, 7-proton), 7.07(1H, dd, J=2.7 Hz, J=8.7 Hz, 6-proton), 4.52(2H, q, J=7.2 Hz, methylene of 2-ethoxy), 3.91(3H, s, 5-methoxy), 1.45(3H, t, J=7.2 Hz, methyl of 2-ethoxy); IR(KBr pellet) 3448, 3157, 2985, 2833, 1709, 1637, 1581, 1531, 1467, 1433, 1398, 1371, 1244, 1201, 1122, 1028, 983, 804 cm⁻¹; MS(EI)247(M⁺), 218(M⁺-HCO), 200, 186, 173, 158, 144, 130, 119. Anal. Calcd(C₁₃H₁₃NO₄)C, H, N.

2-Ethoxycarbonyl-3-formyl-5-methoxy-6-methylindole (18b) Yield 90%; mp 223° C.; TLC (CHCl₃), R_{f}=0.45; ¹HNMR(CDCl₃) δ10.72 (1H, s, 3-formyl), 9.20 (1H, bs, indole proton), 7.81 and 7.21 (2H, 2s, 4,7-proton), 4.51(2H, q, J=7.2 Hz, methylene of 2-ethoxy), 3.93(3H, s, 5-methoxy), 2.34 (3H, s, 6-methyl), 1.45(3H, t, J=7.2 Hz, methyl of 2-ethoxy). IR(KBr pellet) 3435, 3105, 3053, 2993, 2939, 2835, 1718, 1639, 1572, 1531, 1469, 1433, 1390, 1371, 1263, 1205, 1147, 1093, 1060, 962, 866, 812 cm⁻¹; MS(EI)261(M⁺) 232(M⁺-HCO), 214, 200, 187, 172, 158, 144, 133. Anal. Calcd(C₁₄H₁₅NO₄)C, H, N.

3-Formyl-2-methoxycarbonyl-6-methoxyindole (18c) Yield 90%; mp 185° C.; TLC (CHCl₃:acetone 95:5), R_{f}=0.65; ¹HNMR(CDCl₃) δ10.69 (1H, s, 3-formyl), 9.10(1H, bs, indole proton), 8.32(1H,d, J=8.7 Hz, 4-proton), 7.01(1H, dd, J=2.7 Hz, J=8.7 Hz, 5-proton), 6.85(1H, d, J=2.7 Hz, 7-proton), 4.04 and 3.87(6H, 2s, 6-methoxy and ester mrthoxy); IR(KBr pellet) 3311, 3173, 3130, 2953, 2926, 2856, 1712, 1641, 1579, 1444, 1250, 1213, 1161, 1097, 1026, 827 cm⁻¹; MS(EI) 233(M⁺)218, 201, 186, 173, 158, 144. Anal. Calcd(C₁₂H₁₁NO₄)C, H, N.

2-Ethoxycarbonyl-3-formyl-4(or 7)-nitroindole (19) A solution of 2 mmol of 18 was stirred in 25 mL of 70% HNO₃ for 25 min followed by addition of 100 g of ice. The resulting mixture was extracted 3× with 100 mL portions of methylene chloride. The combined extracts were washed with saturated NaHCO₃ and then dried over Na₂SO₄. The product was crystallized from CH₂Cl₂ and hexane.

2-Ethoxycarbonyl-3-formyl-5-methoxy-4-nitroindole (19a) Yield 73%; mp 220° C.; TLC (CHCl₃), R_{f}=0.15; ¹HNMR (CDCl₃) δ10.6(1H, s, 3-formyl), 9.42(1H, bs, indole proton), 7.58 and 7.24(2H, 2d, J=8.7Hz, 6,7-protons), 4.54(2H, q, J=7.2 Hz, methylene of 2-ethoxy), 3.97(3H, s, 5-methoxy), 1.47(3H, t, J=7.2 Hz, methyl of 2-ethoxy). IR(KBr pellet) 3437, 3269, 3080, 2999, 2928, 1689, 1637, 1547, 1523, 1477, 1444, 1390, 1269, 1219, 1184, 1126, 1085, 1012, 869, 833 cm⁻¹; MS(EI)292(M⁺) 274(M⁺-H₂O), 263, 246, 229, 216, 198, 188, 170, 160, 144, 130. Anal. Calcd(C₁₃H₁₂N₂O₆)C, H, N.

2-Ethoxycarbonyl-3-formyl-5-methoxy-6-methyl-4-nitroindole (19b) Yield 50%; mp 198° C; TLC(CHCl₃), R_{f}=0.40; ¹HNMR(CDCl₃) δ10.61(1H, s, 3-formyl), 9.40(1H, bs, indole proton), 7.41(1H, s, 7-proton), 4.53(2H, q, J=7.2 Hz, methylene of 2-ethoxy), 3.91(3H, s, 5-methoxy), 2.48(3H, s, 6-methyl), 1.47(3H, t, J=7.2 Hz, methyl of 2-ethoxy); IR(KBr pellet) 3367, 3105, 2985, 2941, 1726, 1658, 1539, 1464, 1429, 1394, 1253, 1197, 1178, 1016, 977, 864, 808 cm⁻¹; MS(EI)306(M⁺) 288(M⁺-H₂O), 277, 261, 243, 230, 202, 196, 174, 156, 144, 130. Anal. Calcd(C₁₄H₁₄N₂O₆)C, H, N.

3-Formyl-2-methylcarboxyl-6-methoxy-7-nitroindole (19c) Yield 92%; mp 197° C.; TLC(acetone:dichloromethane 20:80), R_{f}=0.80; ¹HNMR(CDCl₃) δ10.74(1H, s, 3-formyl), 10.61(1H, bs, indole proton), 8.71 and 7.15(2H, 2d, J=8.7 Hz, 4,5-protons), 4.12 and 4.08(6H, 2s, 6-methoxyl and 2-ester methyl); IR(KBr pellet)3396, 2960, 2883, 1716, 1662, 1545, 1442, 1411, 1342, 1253, 1192, 1089, 962, 810 cm⁻¹; MS(EI) 278(M⁺)263, 260, 245, 218, 202, 187, 169. Anal. Calcd(C₁₂H₁₁N₂O₆)C, H, N.

2,3-Di(hydroxymethyl)nitroindole (20) To a solution of 100 mg LAH in 15 ml dry THF cooled to −15° C. was added a solution of 0.5 mmol of 19 in 2 mL of dry THF. The solution was stirred at the same temperature for another 5 min and 5 mL of ethyl acetate was added followed by the addition of 2 mL of water. The solid was filtered off and the solution was vacuum dried followed by a flash chromatography using ethyl acetate as the eluent. The product was recrystallized from ethyl acetate and hexane.

2,3-Di(hydroxymethyl)-5-methoxy-4-nitroindole (20a) Yield 40%; mp 127° C.; TLC(ethyl acetate), R_{f}=0.30; ¹HNMR(d₆-DMSO) δ11.4 (1H, bs, indole proton), 7.51 and 7.02(2H, 2d, J=9 Hz, 6, 7-protons), 5.30 and 4.35(2H, 2t, J=5.1 Hz, hydroxyls of 2,3-hydroxymethyls), 4.63 and 4.38(4H, 2d, J=5.1 Hz, methylenes of 2,3 -hydroxymethyls), 3.92(3H, s, 6-methoxyl); IR(KBr pellet) 3348, 2960, 2752, 1633, 1579, 1514, 1464, 1435, 1357, 1321, 1261, 1199, 1103, 1039, 979, 829 cm⁻¹; MS(EI)252(M⁺) 234(M⁺-H₂O), 219, 205, 191, 177, 163, 159, 121. Anal. Calcd(C₁₂H₁₂N₂O₅)C, H, N.

2,3-Di(hydroxymethyl)-5-methoxy-6-methyl-4-nitroindole (20b) Yield 44%; mp 158° C.; TLC(ethyl acetate:methanol 95:5), R_{f}=0.50; ¹HNMR(CDCl₃) δ8.52 (1H, bs, indole proton), 7.30(H, 1s, 7-protons), 4.90 and 4.65(4H, 2d, J=5.7 Hz, methylenes of 2,3-hydroxymethyls), 4.02(3H, s, 5-methoxyl), 2.48(3H, s, 6-methyl), 2.23 and 2.04(2H, 2t, J=5.7 Hz, hydroxyls of 2,3-hydroxymethyls); IR(KBr pellet) 3447, 3385, 2924, 2852, 1637, 1560, 1475, 1429, 1356, 1327, 1278, 1228, 1176, 1101, 1001, 916, 866, 815 cm⁻¹; MS(EI) 266(M⁺) 248(M⁺-H₂O), 231, 219, 215, 201, 187, 162, 158, 143, 130. Anal. Calcd(C₁₂H₁₄N₂O₅)C, H, N.

2,3-Di(hydroxymethyl)-6-methoxy-7-nitroindole (20c) Yield 35%; mp 172° C.; TLC(dichloromethane:methanol 95:5), R_{f}=0.18; ¹HNMR(d₆ -DMSO) δ11.05 (1H, bs, indole proton),7.91 and 7.01(2H, 2d, J=9 Hz, 4, 5-protons), 5.07 and 4.76(2H, 2t, J=6.0 Hz, hydroxyls of 2,3-hydroxymethyls), 4.63 and 4.62(4H, 2d, J=6.0 Hz, methylenes of 2,3-hydroxymethyls), 3.94(3H, s, 6-methoxyl); IR(KBr pellet)3443, 3221, 3013, 2924, 2881, 2852, 1629, 1570, 1510, 1421, 1345, 1300, 1253, 1199, 1091, 1012, 958, 846, 894 cm⁻¹; MS(EI) 252(M⁺), 235(M⁺-OH), 221, 205, 158. Anal. Calcd(C₁₁H₁₂N₂O₅)C, H, N.

2,3-Di(hydroxymethyl)indole-4,7-dione (21) A solution of 0.2 mmol of 20 in 15 mL of methanol and 60 mL of water in the presence of 80 mg 5% Pd on carbon was hydrogenated at 50 psi H₂ for 25 min. The catalyst was filtered off utilizing Celite and the filtrate was combined with a solution consisting of 50 mg KH₂PO₄ and 100 mg Fremy salt in 50 mL of H₂O. The solution was stirred at room temperature for 6 h and then extracted 6× with 50 mL portions of ethyl acetate. The combined extracts were dried over Na₂SO₄ and vacuum dried to a residue that was purified by flash chromatography using ethyl acetate as the eluent. The product was recrystallized from ethyl acetate and hexane.

2,3-Di(hydroxymethyl)-5-methoxyindole-4,7-dione (21a) Yield 15%; mp 208° C.; TLC(ethyl acetate), R_{f}=0.20; ¹HNMR(d₆-DMSO) δ12.8 (1H, bs, indole proton), 5.76(1H, s, 6-proton), 4.99 and 4.63(2H, 2t, J=5.4 Hz, hydroxyls of 2,3-hydroxymethyls), 4.58 and 4.46(4H, 2d, J=5.4 Hz, methylenes of 2,3-hydroxymethyls), 3.95(3H, s, 5-methoxy); IR(KBr pellet) 3425, 2926, 2856, 1647, 1599, 1533, 1465, 1398, 1340, 1249, 1167, 1122, 1037, 880 cm⁻¹; MS(EI) 237(M⁺), 219(M⁺-H₂O), 204, 190, 176, 162, 148, 134, 106. Anal. Calcd(C₁₁H₁₁NO₅)C, H, N.

2,3-Di(hydroxymethyl)-5-methoxy-6-methylindole-4,7-dione (21b) Yield 26%; mp 178° C.; TLC (ethyl acetate:methanol 95:5), R_{f}=0.48; ¹HNMR(CDCl₃) δ9.43 (1H, bs, indole proton), 4.75 and 4.67(4H, 2d, J=6.0 Hz, methylenes of 2,3-hydroxymethyls), 4.02 (3H, s, 5-methoxy), 3.82 and 2.20(2H, 2t, J=6.0 Hz, hydroxyls of 2,3-hydroxymethyls), 1.98(3H, s, 6-methyl); IR(KBr pellet) 3437, 3227, 3122, 2961, 2852, 1693, 1635, 1604, 1492, 1444, 1375, 1303, 1240, 1188, 1103, 1051, 1024, 929 cm⁻¹. MS(EI) 251(M⁺), 233(M⁺-H₂O), 218, 104, 190, 176, 162, 146, 134. Anal. Calcd(C₁₂H₁₃NO₅)C, H, N.

2,3-Di(hydroxymethyl)-6-methoxyindole-4,7-dione (21c) Yield 10%; mp 217° C.; TLC (dichloromethane:methanol 90:10), R_{f}=0.30; ¹HNMR(d₆-DMSO) 612.6 (1H, bs, indole proton), 5.75(1H, s, 5-proton), 5.07 and 4.73(2H, 2t, J=6.0 Hz, hydroxyls of 2,3-hydroxymethyls), 4.60 and 4.47(4H, 2d, J=6.0 Hz, methylenes of 2,3-hydroxymethyls), 3.75(3H, s, 6-methoxy). IR(KBr pellet) 3433, 3173, 3117, 2951, 2876, 1670, 1633, 1597, 1248, 1112, 997, 852 cm⁻¹; MS(EI) 237(M⁺), 219(M⁺-H₂O), 204, 190, 176, 162, 148, 134. Anal. Calcd(C₁₁H₁₁NO₅)C, H, N.

Aziridinyl-2,3-di(hydroxymethyl)indole-4,7-diones (6,7,9a) To 0.2 mmol 21 in 20 mL of methanol was added 0.6 mL of ethylenimine and the resulting mixture stirred at room temperature for 10 h. The solvent was removed and the residue purified by flash chromatography using 20% acetone in ethyl acetate as the eluent. The product was recrystallized from ethyl acetate and hexane.

5-Aziridinyl-2,3-di(hydroxymethyl)indole-4,7-dione (6a) Yield 74%; mp 167° C.; TLC(ethyl acetate:methanol 95:5), R_{f}=0.35; ¹HNMR(d₆-DMSO) δ12.3 (1H, bs, indole proton), 5.78(1H, s, 6-proton), 4.99 and 4.65(2H, 2t, J=5.7 Hz, hydroxyls of 2,3-hydroxymethyls), 4.60 and 4.44(4H, 2d, J=5.7 Hz, methylenes of 2,3-hydroxymethyls), 2.14(4H, s, 5-aziridinyl); IR(KBr pellet) 3445, 3209, 3047, 2926, 2856, 1631, 1579, 1498, 1465, 1361, 1271, 1159, 1095, 1003, 846, 804 cm⁻¹; MS(EI) 248(M⁺), 230(M⁺-H₂O), 215, 201, 189, 174. Anal. Calcd(C₁₂H₁₂N₂O₄)C, H, N.

5-Aziridinyl-2,3-di(hydroxymethyl)-6-methylindole-4,7-dione (7a) Yield 75%; mp 165° C.; TLC(ethyl acetate:methanol 95:5), R_{f}=0.38; ¹HNMR(d₆-DMSO) δ12.3 (1H, bs, indole proton), 4.94 and 4.62(2H, 2t, J=5.1 Hz, hydroxyls of 2,3-hydroxymethyls), 4.58 and 4.40(4H, 2d, J=5.1 Hz, methylenes of 2,3-hydroxymethyls), 2.23(4H, s, 5-aziridinyl), 1.92(3H, s, 6-methyl). IR(KBr pellet)3404, 3209, 2949, 2879, 1626, 1587, 1562, 1500, 1458, 1377, 1350, 1248, 1188, 1149, 1103, 1020, 995, 819 cm⁻¹; MS(EI) 262(M⁺), 247(M⁺-CH₃), 244(M⁺-H₂O), 229, 215, 203. Anal. Calcd(C₁₃H₁₄N₂O₄)C, H, N.

6-Aziridinyl-2,3-di(hydroxymethyl)indole-4,7-dione (9a) Yield 52%; mp 175° C.; TLC (ethyl acetate:methanol 95:5), R_{f}=0.38; ¹HNMR(d₆-DMSO) 612.4 (1H, bs, indole proton), 5.76(1H, s, 5-proton), 5.04 and 4.72(2H, 2t, J=5.7 Hz, hydroxyls of 2,3-hydroxymethyls), 4.57 and 4.46(4H, 2d, J=5.7 Hz, methylenes of 2,3-hydroxymethyls), 2.14(4H, s, 6-aziridinyl); IR(KBr pellet) 3394, 3232, 3038, 2928, 2874, 1660, 1610, 1500, 1392, 1275, 1132, 1020, 879 cm⁻¹; MS(EI) 248(M⁺), 230, 202, 189, 174, 160, 146. Anal. Calcd(C₁₂H₁₂N₂O₄)C, H, N.

5-Aziridinyl-2,3-di(acetoxymethyl)indole-4,7-dione (6e) To a solution of 0.2 mmol of 6a in 5 mL of CH₂Cl₂ and 1 mL of acetone containing 50 mg of DMAP was added 100λ acetic anhydride. The solution was stirred at room temperature for 5 min and directly flash chromatographed using ethyl acetate as the eluent. The eluted solution containing the product was washed with NaHCO₃, dried over Na₂SO₄, and vacuum dried. The product was recrystallized from ethyl acetate and hexane: Yield 82%; mp 167° C.; TLC (dichloromethane:methanol 95:5), R_{f}−0.6; ¹HNMR(CDCl₃) δ8.8(1H, bs, indole proton), 5.72(1H, s, 6-proton), 4.63 and 4.46(4H, 2s, methylenes of 2,3-hydroxymethyls), 2.24(4H, s, 5-aziridinyl), 2.10 and 2.08(6H, 2s, 2,3-acetyl methys); IR(KBr pellet) 3447, 3420, 3194, 3047, 2966, 1739, 1674, 1628, 1570, 1506, 1475, 1377, 1246, 1165, 1145, 1112, 1033, 977, 949, 850, 804 cm⁻¹; MS(EI) 332(M⁺), 290(M⁺-acetyl), 279, 261, 243, 226, 217, 188. Anal. Calcd(C₁₆H₁₆N₂O₆)C, H, N.

2-Ethoxycarbonyl-3-formyl-1,6-dimethyl-5-methoxy-4-nitroindole (22) To 19b 0.2 mmol in 20 mL acetone with 500 mg KOH was added 1.0 mL MeI and stirred at room temperature for 1 h and neutralized it with HCl and extracted 4× with 30 mL portions of CH₂Cl₂. The combined extracts was dried with Na₂SO₄ and dried followed by chromatography using CH₂Cl₂ as the eluent. The product was crystallized from CH₂Cl₂ and hexane. Yield 55%; mp 148° C; TLC(CHCl₃), R_{f}=0.55; ¹HNMR(CDCl₃) δ10.42(1H, s, 3-formyl), 7.40(H, s, 7-proton), 4.52(2H, q, J=7.2 Hz, methylene of ethoxy), 4.06 and 3.91(6H, 2s, 1 methyl and 5-methoxyl), 2.51(3H, s, 6-methyl), 1.46(3H, t, J=7.2 Hz, methyl of ethoxy); IR(KBr pellet) 3435, 2999, 2930, 1712, 1666, 1545, 1475, 1404, 1238, 1161, 1031, 979, 908, 779 cm⁻¹; MS(EI) 320(M⁺), 307, 287, 275, 244, 216. Anal. Calcd(C₁₅H₁₆N₂O₆)C, H, N.

2,3-Di(hydroxymethyl)-1,6-dimethyl-5-methoxy-4-nitro-indole (23) Follow the same procedure for the synthesis of 21: Yield 55%; mp 197° C.; TLC(EtOAc:MeOH 95:5), R_{f}=0.65. ¹HNMR(d₆-DMSO) δ7.58(1H, 1s, 7-protons), 5.13 and 4.44(2H, 2t, J=5.4 Hz, hydroxyls of 2,3-hydroxymethyl), 4.61 and 4.42(4H, 2d, J=5.4 Hz, methylenes of 2,3-hydroxymethyls), 3.77 and 3.75(6H, 2s, 1-methyl and 5-methoxy), 2.40(3H, s, 6-methyl). IR(KBr pellet) 3447, 2945, 2891, 1637, 1525, 1477, 1377, 1329, 1174, 1124, 993, 854, 765 cm⁻¹. MS(EI) 280(M⁺), 262, 245, 229, 215, 186, 173. Anal. Calcd(C₁₃H₁₆N₂O₅)C, H, N.

2,3-Di(hydroxymethyl)-1,6-dimethyl-5-methoxyindole-4,7-dione (24) Yield 16%; mp 160° C.; TLC (ethyl acetate:methanol 95:5) R_{f}=0.56; ¹H NMR(DMSO- d₆) δ5.13 and 4.67(2H, 2t, J=5.4 Hz, hydroxyls of 2,3-hydroxymethyls), 4.59 and 4.54(4H, 2d, J=5.4 Hz, methylenes of 2,3-hydroxymethyls), 3.90 and 3.87(6H, 2s, 1-methyl and 5-methoxy), 1.85(3H, s, 6-methyl); IR(KBr pellet)3335, 3194, 2943, 2852, 1682, 1643, 1506, 1464, 1313, 1122, 1008, 989, 900, 740 cm⁻¹; MS(EI)265(M⁺), 247(M⁺-H₂O), 232, 218, 204, 190, 176, 148, 132, 120. Anal. Calcd(C₁₃H₁₅NO₅)C, H, N.

5-Aziridinyl-2,3-di(hydroxymethyl)-1,6-dimethylindole-4,7-dione (7c) Yield 50%; mp 165° C.; TLC (ethyl acetate:methanol 95:5), R_{f}=0.48; ¹HNMR(d₆-DMSO) δ5.08 and 4.59(2H, 2t, J=5.4 Hz, hydroxyls of 2,3-hydroxymethyls), 4.58 and 4.52(4H, 2d, J=5.4 Hz, methylenes of 2,3-hydroxymethyls), 3.89(3H, s, 1-methyl), 2.23(4H, s, 5-aziridinyl), 1.93(3H, s, 6-methyl); IR(KBr pellet)3435, 3335, 3057, 2958, 2852, 1666, 1631, 1508, 1336, 1151, 1043, 989, 875 cm⁻¹; MS(EI) 276(M⁺), 258, 243, 229,214, 202, 188. Anal. Calcd(C₁₄H₁₆N₂O₄)C, H, N.

2-Ethoxycarbonyl-3-(hydroxymethyl)nitroindole (25) To a solution of 2 mmol 19 in 100 mL of MeOH was added 1 g of NaBH₄ and the solution was stirred at room temperature for 20 min followed by the addition of 200 mL of water. The solution was then extracted 5× with 100 mL portions of chloroform. The extract was dried over Na₂SO₄ and vacuum dried to a residue. The product was crystallized from CH₂Cl₂ and hexane.

2- Ethoxycarbonyl-3-hydroxymethyl-5-methoxy-4-nitroindole (25a) Yield 90%; mp 165° C.; TLC (chloroform:acetone 80:20), R_{f}=0.40; ¹HNMR(CDCl₃) δ10.45(1H, bs, indole proton), 7.54 and 7.28(2H, 2d, J=8.7 Hz, 6, 7-protons), 5.12(2H, d, J=6.6 Hz, 3-methylene), 5.00(1H, t, J=6.6 Hz, 3-hydroxyl), 4.54(2H, q, J=7.2 Hz, methylene of ethoxy), 4.01(3H, s, 5-methoxy), 1.48(3H, t, J=7.2 Hz, methyl of ethoxy); IR(KBr pellet) 3562, 3452, 3313, 2993, 2949, 2849, 1682, 1533, 1475, 1384, 1255, 1180, 1120, 1012, 827 cm⁻¹; MS(EI)294(M⁺), 276, 248, 231, 212, 186. Anal. Calcd(C₁₃H₁₄N₂O₆)C, H, N.

2- Ethoxycarbonyl-3-hydroxymethyl-5-methoxy-6-methyl-4-nitroindole (25b) Yield 90%; mp 147° C.; TLC (chloroform:acetone 80:20), R_{f}=0.50; ¹HNMR(CDCl₃) δ8.91(1H, bs, indole proton), 7.35(1H, s, 7-proton), 4.97(2H, d, J=6.3 Hz, 3-methylene), 4.45(2H, q, J=7.2 Hz, methylene of ethoxy), 3.90(3H, s, 5-methoxy), 2.49(1H, t, J=6.3 Hz, 3-hydroxyl), 2.48(3H, s, 6-methyl), 1.44(3H, t, J=7.2 Hz, methyl of ethoxy); IR(KBr pellet) 3431, 3238, 3001, 2957, 2889, 1718, 1701, 1527, 1465, 1261, 1188, 1155, 1006, 779 cm⁻¹. MS(EI)308(M⁺), 291(M⁺-OH), 279, 261, 245, 226, 215, 200, 187, 174. Anal. Calcd(C₁₄H₁₆N₂O₆)C, H, N.

3-Hydroxymethyl-6-methoxy-2-methoxycarbonyl-7-nitroindole (25c) Yield 93%; mp 150° C.; TLC (chloroform:methanol 90:10), R_{f}=0.35; ¹HNMR(CDCl₃) δ10.25(1H, bs, indole proton), 8.03 and 7.01(2H, 2d, J=9.3 Hz, 4,5-protons), 5.07(2H, d, J=6.9 Hz, 3-methylene), 4.90(1H, t, J=6.9 Hz, 3-hydroxyl), 4.10 and 4.02(6H, 2s, 6-methoxy and ester methyl); IR(KBr pellet) 3433, 2920, 2850, 1736, 1629, 1560, 1521, 1464, 1253, 1180, 1091, 970, 804 cm⁻¹; MS(EI)280(M⁺), 263(M⁺-OH), 247, 231, 219, 200, 172, 157. Anal. Calcd(C₁₂H₁₂N₂O₆)C, H, N.

2- Ethoxycarbonyl-3-(hydroxymethyl)indole-4,7-dione (26) A solution of 2 mmol of 25 in 80 mL of methanol with 300 mg 5% Pd on carbon was reduced under 50 psi of H₂ for 25 min and the catalyst was filtered off using Celite. The solvent was vacuum dried and the residue was dissolved in 5 mL of acetone, which was then mixed with a solution of 800 mg KH₂PO₄ and 1.6 g Fremy salt in 200 mL of H₂O. The solution was stirred at room temperature for 5 h and extracted 5× with 200 mL of methylene chloride. The extract was dried and purified by a flash chromatography using 10% acetone in methylene chloride as the eluent. The product was precipitated from CH₂Cl₂ and hexane.

2- Ethoxycarbonyl-3-hydroxymethyl-5-methoxyindole-4,7-dione (26a) Yield 64%; mp 213° C.; TLC (chloroform:methanol 90:10), R_{f}=0.30; ¹HNMR(CDCl₃) δ9.77(1H, bs, indole proton), 5.85(1H, s, 6-proton), 5.07(2H, d, J=7.2 Hz, 3-methylene), 4.41(2H, q, J=7.2 Hz, methylene of ethoxy), 4.22(1H, t, J=7.2 Hz, 3-hydroxyl), 3.89 (3H, s, 5-methoxy), 1.41(3H, t, J=7.2 Hz, methyl of ethoxy); IR(KBr pellet)3433, 3122, 3072, 2989, 2924, 2785, 1712, 1649, 1597, 1560, 1487, 1467, 1340, 1280, 1161, 1031, 844 cm⁻¹; MS(EI)279 (M⁺),261, 250, 233, 218, 205, 190, 177. Anal. Calcd(C₁₃H₁₃NO₆)C, H, N.

2-Ethylcarboxyl-3-hydroxymethyl-5-methoxy-6-methylindole-4,7-dione (26b) Yield: 41%. MP: 183° C. TLC: (CHCl₃:MeOH 90:10), R_{f}=0.45. ¹HNMR(CDCl₃) δ9.75(1H, bs, indole proton), 5.05(2H, d, J=7.2 Hz, 3-methylene), 4.41(2H, q, J=7.2 Hz, methylene of ethoxy), 4.34(1H, t, J=7.2 Hz, 3-hydroxyl), 4.08(3H, s, 5-methoxy), 2.01(3H, s, 6-methyl), 1.41(3H, t, J=7.2 Hz, methyl of ethoxy); IR(KBr pellet)3435, 3211, 3063, 2991, 2920, 2852, 1710, 1647, 1606, 1496, 1294, 1232, 1112, 1074, 1033, 866 cm⁻¹; MS(EI) 293(M⁺), 275, 264, 247, 232, 219, 204, 186. Anal. Calcd(C₁₄H₁₅NO₆)C, H, N.

3-(Hydroxymethyl)-6-methoxy-2-(methoxycarbonyl)indole-4,7-dione (26c) Yield 42%; mp 150° C.; TLC (chloroform:methanol 90:10), R_{f}=0.35; ¹HNMR(CDCl₃) δ9.70(1H, bs indole proton), 5.91(1H, s, 5-proton), 5.04(2H, d, J=7.8 Hz, 3-methylene), 4.59(1H, t, J=7.8 Hz, 3-hydroxyl), 3.96 and 3.88(6H, 2s, 6-methoxy and ester methyl); IR(KBr pellet)3431, 3238, 3001, 2957, 2889, 1718, 1701, 1527, 1465, 1398, 1261, 1188, 1155, 1006, 779 cm⁻¹; MS(EI)265(M⁺), 250(M⁺-Me), 233, 218, 205, 190, 162, 148, 120. Anal. Calcd(C₁₂H₁₁NO₆)C, H, N.

Aziridinyl-2-ethoxycarbonyl-3-(hydroxymethyl)indole-4,7-dione. To a solution of 1 mmol of product above in 50 mL of methanol was added 1 mL of aziridine and the reaction stirred at room temperature for 20 h. The solvent was evaporated ad the residue was purified by flash chromatography on silica gel using 10% acetone in CH₂Cl₂ as the eluent. The product was recrystallized from CH₂Cl₂ and hexane.

5-Aziridinyl-2-ethylcarboxyl-3-(hydroxymethyl)indole-4,7-dione (6c) Yield 86%; mp 170° C. TLC: (chloroform:methanol 90:10), R_{f}=0.32. ¹HNMR(CDCl₃) δ9.78 (1H, bs, indole proton), 5.96 (1H, s, 6-proton), 5.07(2H, d, J=7.2 Hz, 3-methylene), 4.41(2H, q, J=7.2 Hz, methylene of ethoxy), 4.34(1H, t, J=7.2 Hz, 3-hydroxyl), 2.30 (4H, s, 5-aziridinyl), 1.41(3H, t, J=7.2 Hz, methyl of ethoxy); IR(KBr pellet)3450, 3259, 3024, 2899, 2862, 1703, 1656, 1581, 1550, 1491, 1363, 1278, 1240, 1149, 1028, 842 cm⁻¹; MS(EI) 290(M⁺), 279, 260, 244, 233, 226, 215, 199, 188, 171. Anal. Calcd(C₁₄H₁₄N₂O₅)C, H, N.

5-Aziridinyl-2-ethylcarboxyl-3-hydroxymethyl-6-methylindole-4,7-dione (7b) Yield 76%; mp 197° C.; TLC (chloroform:methanol 90:10), R_{f}=0.40; ¹HNMR(CDCl₃) δ9.61(1H, bs, indole proton), 5.04(2H, d, J=7.2 Hz, 3-methylene), 4.45(2H, q, J=7.2 Hz, methylene of ethoxy), 4.38(1H, t, J=7.2 Hz, 3-hydroxyl), 2.39(4H, s, 5-aziridinyl), 2.10(3H, s, 6-methyl), 1.41(3H, t, J=7.2 Hz, methyl of ethoxy); IR(KBr pellet) 3448, 3252, 3088, 2997, 2922, 2854, 1709, 1651, 1587, 1550, 1492, 1377, 1209, 1261, 1176, 1147, 1035, 997, 962, 871 cm⁻¹; MS(EI)304(M⁺), 286(M⁺-H₂O), 275, 258, 240, 229, 212, 202. Anal. Calcd(C₁₅H₁₆N₂O₅)C, H, N.

6-Aziridinyl-3-(hydroxymethyl)-2-methylcarboxylindol-4,7-dione (9b) Yield 52%; mp 258° C.; TLC (chloroform:methanol 90:10), R_{f}=0.45; ¹HNMR(CDCl₃) δ9.78(1H, bs, indole proton), 6.02(1H, s, 5-proton), 5.02(2H, d, J=6.9 Hz, 3-methylene), 4.59(1H, t, J=6.9 Hz, 3-hydroxyl), 3.93 (3H, s, ester methyl), 2.28(4H, s, 6-aziridinyl); IR(KBr pellet)3420, 3257, 3074, 2997,2958,2922,2852, 1705, 1674, 1626, 1587, 1545, 1267, 1157, 1078, 1030, 952, 868, 804 cm⁻¹; MS(EI)276(M⁺), 244(M⁺-CO), 229, 216, 188, 160, 132. Anal. Calcd(C₁₃H₁₂N₂O₅)C, H, N.

3-Acetoxymethyl-5-aziridinyl-2-ethoxycarbonylindole-4,7-dione (6d) Follow the procedure described for the acetylation of 6e. Yield 73%; mp 185° C.; TLC(chloroform:acetone 90:10), R_{f}=0.45; ¹HNMR(CDCl₃) δ9.76(1H, bs, indole proton), 5.96(1H, s, 6-proton), 5.54(2H, s, 3-methylene), 4.42(2H, q, J=7.2 Hz, methylene of ethoxy), 2.29 (4H, s, 5-aziridinyl), 2.07(3H, s, 3-acetoxymethyl), 1.39(3H, t, J=7.2 Hz, methyl of ethoxy); IR(KBr pellet)3445, 3234, 2995, 2920, 2852, 1726, 1701, 1676, 1583, 1556, 1500, 1363, 1280, 1244, 1153, 1024, 819 cm⁻¹; MS(EI) 332(M⁺), 318, 290, 279, 260, 244, 217. Anal. Calcd(C₁₆H₁₆N₂O₆)C, H, N.

2-Ethoxycarbonyl-5-methoxy-3-methyl-4-nitroindole (27a) To 1.17 g (5 mmol) 2-ethylcarboxyl-5-methoxy-3-methylindole {Allen, M. S., Hamaker, L. K., Laloggia, A. J., and Cook, J. M., Entry Into 6-Methoxy-D(+)-Tryptophans-Stereospecific Synthesis of 1-Benzenesulfonyl-6-Methoxy-D(+)-Tryptophan Ethyl-Ester, Synth. Commun., 1992, 22, 2077-2102} in 180 mL of CH₂Cl₂, cooled by dry ice bath at −20° C., was added 0.5 mL of 70% HNO₃ and the solution stirred for 10 min. The solution was then neutralized with NaHCO₃ aqueous solution and extracted 4× with 50 mL portions of methylene chloride. The extracts were dried over Na₂SO₄ and vacuum dried to a residue that was purified by flash chromotography on ilica gel using CH₂Cl₂ as the eluent: 880 mg (69%)Yield; mp 175-177° C.; TLC (CHCl₃), R_{ƒ}=0.5; ¹HNMR(CDCl₃) δ8.81(1H, bs, indole proton), 7.45 and 7.12(2H, d, J=9 Hz, 6- and 7-protons), 4.44(2H, q, J=7.5 Hz, methylene of ethoxy), 3.94 (3H, s, 5-methoxy), 2.46 (3H, s, 3-methyl), 1.43 (3H, t, J=7.5, methyl of ethoxy); IR(KBr pellet) 3423, 3340, 2933, 2847, 1680, 1628, 1541, 1475, 1384, 1251, 1182, 1120, 1049, 1016, 796 cm⁻¹; MS(EI) 278(M⁺), 261(M⁺-OH), 249, 232, 215, 203, 187, 174, 156. Anal. Calcd (C₁₃H₁₄N₂O₅)C, H, N.

2- Ethoxycarbonyl-1,3-dimethyl-4-nitro-indole (28) To 2 mmol of 27 in 20 mL of acetone containing 400 mg of KOH was added 1.0 mL of MeI and the mixture stirred at room temperature for 1 h. The reaction mixture was then neutralized with HCl and extracted 4× with 30 mL portions of CH₂Cl₂. The combined extracts were dried over Na₂SO₄ and evaporated to a residue, that was purified chromatography on silica gel using CH₂Cl₂ as the eluent. The purified product was recrystallized from CH₂Cl₂ and hexane.

2-Ethoxycarbonyl-5-methoxy-1,3-dimethyl-4-nitroindole (28a) Yield˜100%; mp 120-122° C.; TLC (CHCl₃), R_{f}=0.45; ¹HNMR(CDCl₃) δ7.42 and 7.13(2H, d, J=9.3Hz, 6 and 7 protons), 4.42(2H, q, J=7.2 Hz, 2-methylene of ethyl), 3.98 and 3.94(6H, 2s, 1-methyl and 5-methoxy), 2.40(3H, s, 3-methyl), 1.43(3H, t, J=7.2, 2-methyl of ethyl); IR(KBr pellet) 3441, 3003, 2947, 2912, 1709, 1637, 1527, 1465, 1375, 1236, 1165, 1112, 1045, 912, 794 cm⁻¹; MS(EI)292(M⁺), 275(M⁺-OH), 263, 244, 216, 188, 163, 149. Anal. Calcd(C₁₄H₁₆N₂O₅)C, H, N.

2-Ethoxycarbonyl-1,3,5-trimethyl-4-nitroindole (28b) Yield 90%; mp 65-68° C.; TLC (CHCl₃), R_{f}=0.50; ¹HNMR(CDCl₃) δ7.38 and 7.18(2H, d, J=9 Hz, 6 and 7 protons), 4.46(2H, q, J=7.5 Hz, 2-methylene of ethoxy), 3.99(3H, s, 1-methyl), 2.42 and 2.39(6H, 2s, 3,5-methyls), 1.43(3H, t, J=7.5, 2-methyl of ethoxy); IR(KBr pellet) 3338, 2978, 2928, 2854, 1699, 1521, 1373, 1290, 1251, 1155, 1085, 1014, 802 cm⁻¹; MS(EI)276(M⁺), 259(M⁺-OH), 247, 229, 213, 202,174,157,142,131, 115. Anal. Calcd(C₁₄H₁₆N₂O₄)C, H, N.

2-Ethoxycarbonyl-3-(hydroxymethyl)-4-nitroindole (29) To 1.50 mmol 28 in 15 mL of CCl₄ at reflux was added 258 mg of NBS and 26 mg AIBN. The reaction was refluxed for 1.5 h with additional AIBN was added every 30 min (13 mg×3). Hexane was added to the cooled reaction mixture in order to precipitate the crude product as light yellow solid. A solution of the crude product above in 25 mL of THF, 20 mL of H₂O and 4 mL of HCl was stirred at room temperature for 1.5 h. This mixture was neutralized with saturated NaHCO₃ solution and then extracted 3× with 50 mL portions of CH₂Cl₂. The extracts were dried over Na₂SO₄ and concentrated to a residue. The product was recrystallized from CH₂Cl₂ and hexane.

2-Ethoxycarbonyl-3-hydroxymethyl-5-methoxy-1-methyl-4-nitro-indole (29a) Yield: 34%. MP: 120-122° C. TLC: (CHCl₃:MeOH 9:1), R_{f}=0.55. ¹HNMR(CDCl₃) δ7.48 and 7.17(2H, d, J=8.7 Hz, 6 and 7 protons), 4.87(2H, d, J=6.6 Hz, 3-hydroxymethyl), 4.47(2H, q, J=7.2 Hz, 2-methylene of ethyl), 4.01 and 3.96(6H, 2s, 1-methyl and 5-methoxyl), 2.31(1H, t, J=6.6 Hz, 3-hydroxy), 1.46(3H, t, J=7.2, 2-methyl of ethyl). IR(KBr pellet) 3497, 3412, 3232, 2947, 1685, 1621, 1609, 1534, 1287, 1174, 1000, 823 cm⁻¹. MS(EI) 308(M⁺), 291(M⁺-OH), 261, 244, 235, 216, 201, 177, 163,149, 105. Anal. Calcd(C₁₄H₁₆N₂O₆)C, H, N.

2-Ethoxycarbonyl-3-(hydroxymethyl)-5-methyl-4-nitroindole (29b) Yield 80%; mp 134-136° C.; TLC (chloroform:methanol 9:1), R_{f}=0.45; ¹HNMR(CDCl₃) δ9.08 (1H, bs indole proton), 7.46 and 7.23(2H, d, J=9 Hz, 6 and 7 protons), 5.01(2H, d, J=6 Hz, 3-hydroxymethyl), 4.46(2H, q, J=7.5 Hz, 2-methylene of ethoxy), 2.48 (3H, s, 5-methyl), 2.35(1H, t, J=6 Hz, 3-hydroxy), 1.47(3H, t, J=7.5, methyl of ethoxy); IR(KBr pellet) 3447, 3194, 2980, 2928, 1718, 1678, 1527, 1465, 1365, 1265, 1180, 1116, 989, 808 cm⁻¹; MS(EI)278(M⁺), 261(M⁺-OH), 245, 231, 215, 186, 171, 149. Anal. Calcd (C₁₃H₁₄N₂O₅)C, H, N.

2-Ethoxycarbonyl-3-(hydroxymethyl)-1,5-dimethyl-4-nitroindole (29c) Yield 31%; mp 113-115° C.; TLC (chloroform:methanol 9:1), R_{f}=0.50; ¹HNMR(CDCl₃) δ7.43 and 7.23(2H, d, J=9 Hz, 6 and 7 protons), 4.88(2H, d, J=6 Hz, 3-hydroxymethyl), 4.48(2H, q, J=7.5 Hz, methylene of ethoxy), 4.01 and 2.48(6H, 2s, 1 and 5-methyl), 2.35(1H, t, J=6 Hz, 3-hydroxy), 1.46(3H, t, J=7.5 Hz, methyl of ethoxy); IR(KBr pellet) 3489, 3425, 3244, 2928, 1685, 1637, 1618, 1525, 1263, 1168, 1014, 810 cm⁻¹; MS(EI)292(M⁺), 275(M⁺-OH), 245, 228, 219, 200, 185, 172. Anal. Calcd(C₁₄H₁₆N₂O₅)C, H, N.

2,3-Di(hydroxymethyl)-4-nitroindole (30) To a solution of 0.3 mmol of 29 in 4 mL of dry THF cooled to −10° C. was added 60 mg of LAH and the solution was stirred for 5 min. The unreacted LAH was quenched by gradually adding 5 mL of ethyl acetate followed by 2 mL of H₂O. The ethyl acetate layer was separated and the aqueous layer was washed 5× with 20 mL portions of ethyl acetate. The combined extracts were dried over Na₂SO₄ and concentrated to a residue. Purification was carried out by flash chromatography on silica gel using ethyl acetate as the eluent.

2,3-Di(hydroxymethyl)-5-methoxy-1-methyl-4-nitroindole (30a) Yield 66%; mp 163-165° C.; TLC (chloroform:methanol 9:1), R_{f}=0.35; ¹HNMR(CDCl₃) δ7.42 and 7.02 (2H, 2d, J=8.7 Hz, 6 and 7 protons), 4.85 and 4.69(4H, 2d, J=6 Hz, methylenes of 2 and 3-hydroxymethyls), 3.95 and 3.84(6H, 2s, 1-methyl and 5-methoxy), 2.33 and 2.19(2H, 2t, J=6 Hz, 2 and 3-hydroxys); IR(KBr pellet) 3425, 2924, 2847, 1628, 1521, 1475, 1421, 1365, 1300, 1263, 1105, 1062, 1001, 974, 891 cm⁻¹; MS(EI)266(M⁺), 249(M⁺-OH), 237, 208, 194, 189,165, 121. Anal. Calcd(C₁₂H₁₄N₂O₅)C, H, N.

2,3-Di(hydroxymethyl)-5-methyl-4-nitroindole (30b) Yield 60%; mp 132-134° C.; TLC (chloroform:methanol 9:1), R_{f}=0.3; ¹HNMR(d₆-DMSO) δ11.5(1H, bs, indole proton), 7.45 and 7.03(2H, d, J=9 Hz, 6 and 7 protons), 5.31 and 4.38(2H, 2t, J=6 Hz, 2 and 3-hydroxys), 4.64 and 4.40(4H, 2d, J=6 Hz, 2 and 3-methylenes of hydroxymethyls), 2.34(3H, s, 5-methyl); IR(KBr pellet) 3394, 2995, 2926, 1637, 1510, 1475, 1363, 1321, 1151, 1014, 974 cm⁻¹; MS(EI)236(M⁺), 219(M⁺-OH), 207, 177, 163, 149, 105. Anal. Calcd(C₁₁H₁₂N₂O₄)C, H, N.

2,3-Di(hydroxymethyl)-1,5-dimethyl-4-nitroindole (30c) Yield 59%; mp 119-121° C.; TLC: (chloroform:methanol 9:1), R_{f}=0.34; ¹HNMR(d₆-DMSO) δ7.63 and 7.16(2H, d, J=9 Hz, 6 and 7 protons), 5.18 and 4.40(2H, 2t, J=6 Hz, 2 and 3-hydroxys), 4.63 and 4.45(4H, 2d, J=6 Hz, 2 and 3-methylenes of hydroxymethyls), 3.78 and 2.34(6H, 2s, 1 and 5-methyl); IR(KBr pellet) 3425, 2972, 2925, 1647, 1518, 1465, 1373, 1342, 1300, 1182, 1069, 1055, 989, 802 cm⁻¹; MS(EI)250(M⁺), 233(M⁺-OH), 221, 191, 177, 163,149, 105. Anal. Calcd(C₁₂H₁₄N₂O₄)C, H, N.

2,3-Di(hydroxymethyl)indole-4,7-dione (31) A solution of 0.5 mmol product above with 295 mg 5% Pd—C in 40 mL methanol was reduced under 50 Psi H₂ for 30min. The catalyst was Celite filtered off and the filtrate was vacuum dried. The solid residue was dissolved in 4 mL of acetone and mixed with a solution of 250 mg KH₂PO₄ and 500 mg Fremy salt in 25 mL of water. The solution was stirred at room temperature for 4 h and extracted with 5× with 50 mL portions of ethyl acetate. The extracts were dried over Na₂SO₄ and vacuum dried and then purified by flash column chromatography on silica gel using ethyl acetate as the eluent.

2,3-Di(hydroxymethyl)-5-methoxy-1-methylindole-4,7-dione (31a) Yield 25%; mp 191-193° C; TLC(chloroform:methanol 9:1), R_{f}=0.45; ¹HNMR(CDCl₃) δ6.23(1H, s, 6-proton), 4.70 and 4.65(4H, 2d, J=6 Hz, 2 and 3-methylenes of hydroxymethyls), 4.11 and 1.98(2H, 2t, J=6 Hz, 2 and 3-hydroxy of hydroxymethyls), 4.04 and 3.84(6H, 2s, 1-methyl and 5-methoxy); IR(KBr pellet) 3465, 3332, 2945, 2931, 1649, 1537, 1419, 1374, 1233, 950, 811 cm⁻¹; MS(EI)251(M⁺), 233(M⁺-H₂O), 218, 204, 190, 176, 162, 120. Anal. Calcd(C₁₂H₁₃NO₅)C, H, N.

2,3-Dihydroxymethyl-5-methyl-indol-4,7-dione (31b) Yield 52%; mp 181-183° C.; TLC (chloroform:methanol 9:1), R_{f}=0.24; ¹HNMR(d₆-DMSO) δ12.4 (1H, bs, 1-indole proton), 6.44(1H, q, J=1.5 Hz, 6-proton), 5.03 and 4.66(2H, 2t, J=6 Hz, 2 and 3-hydroxys of hydroxymethyls), 4.59 and 4.46(4H, 2d, J=6 Hz, 2 and 3-methylenes of hydroxymethyls), 1.94(3H, s, 5-methyl); IR(KBr pellet) 3423, 3101, 2953, 2930, 1707, 1523, 1465, 1384, 1253, 1159, 1109, 991, 806 cm⁻¹; MS(EI)221(M⁺), 203(M⁺-H₂O), 190, 174, 147, 118, 107. Anal. Calcd(C₁₁H₁₁NO₄)C, H, N.

2,3-Dihydroxymethyl-1,5-dimethyl-indol-4,7-dione (31c) Yield 32%; mp 136-138° C.; TLC (chloroform:methanol 9:1), R_{f}=0.50; ¹HNMR(CDCl₃) δ6.40(1H, q, J=1.5 Hz, 6-proton), 4.72 and 4.67(4H, 2d, J=6 Hz, 2 and 3-methylenes of hydroxymethyls), 4.04 and 1.94(2H, 2t, J=6 Hz, 2 and 3-hydroxy of hydroxymethyls), 4.04(3H, s, 1-methyl), 2.34(3H, s, 5-methyl); IR(KBr pellet) 3439, 3377, 2955, 2928, 1643, 1608, 1508, 1465, 1377, 1242, 1020, 993, 802 cm⁻¹; MS(EI)235(M⁺), 217(M⁺-H₂O), 202, 188, 174, 161, 146, 133. Anal. Calcd(C₁₂H₁₃NO₄)C, H, N.

5-Aziridinyl-2,3-di(hydroxymethyl)-1-methylindole-4,7-dione (6b) Yield 87%; mp 145-147° C.; TLC (chloroform:methanol 9:1); R_{f}=0.40; ¹HNMR(CDCl₃) δ5.82(1H, s, 6-hydrogen), 4.38 and 2.22(2H, 2t, J=5.4 Hz, 2 and 3-hydroxys of hydroxymethyls), 4.71 and 4.66(4H, 2d, J=5.4 Hz, 2 and 3-methylenes of hydroxymethyls), 4.01(3H, s, 1-methyl), 2.22(4H, s, 5-aziridinyl); IR(KBr pellet) 3539, 3362, 2954, 2919, 1683, 1623, 1514, 1472, 1376, 1250, 1031, 810 cm⁻¹; MS(EI)262(M⁺), 244(M⁺-H₂O), 229, 215, 201, 169, 159, 146. Anal. Calcd(C₁₃H₁₄N₂O₄)C, H, N.

6-Aziridinyl-2,3-di(hydroxymethyl)-5-methylindole-4,7-dione (8a) Yield 33%; mp 193-194° C.; TLC (chloroform:methanol 9:1), R_{f}=0.20; ¹HNMR(d₆-DMSO) δ12.3(1H, bs, 1-indole proton), 5.03 and 4.70(2H, 2t, J=5.4 Hz, 2 and 3-hydroxys of hydroxymethyls), 4.57 and 4.44(4H, 2d, J=5.4 Hz, 2 and 3-methylenes of hydroxymethyls), 2.26(4H, s, 6-aziridinyl), 1.91(3H, s, 5-methyl); IR(KBr pellet) 3465, 3097, 2943, 2928, 1617, 1543, 1465, 1375, 1234, 1108, 994, 812 cm⁻¹; MS(EI)262(M⁺), 244(M⁺-H₂O), 229, 215, 201, 188, 169, 159, 146, 134, 108. Anal. Calcd(C₁₃H₁₄N₂O₄)C, H, N.

6-Aziridinyl-2,3-di(acetoxymethyl)-5-methylindole-4,7-dione (8b) Yield 88%; mp 135-137° C.; TLC (chloroform:acetone 9:1), R_{f}=0.60; ¹HNMR(CDCl₃) δ9.4(11H, bs, 1-indole proton), 5.31 and 5.19(4H, 2s, methylenes), 2.28(4H, s, 6-aziridinyl), 2.10, 2.08 and 2.05(9H, 3s, methyls); IR(KBr pellet) 3390, 3088, 2956, 2917, 1635, 1498, 1364, 1211, 1134, 940, 825 cm⁻¹; MS(EI)346(M⁺), 303(M⁺-acetate), 287, 244, 226, 215. Anal. Calcd(C₁₇H₁₈N₂O₆)C, H, N.

6-Aziridinyl-2,3-di(hydroxymethyl)-1,5-dimethylindol-4,7-dione (8c) Yield 69%; mp 140-141° C.; TLC (chloroform:methanol 9:1), R_{f}=0.46; ¹HNMR(CDCl₃) δ4.66 and 4.63(4H, 2d, J=5.4 Hz, methylenes), 4.49 and 2.15(2H, 2t, J=5.4 Hz, hydroxyls), 3.99(3H, s, 1-methyl), 2.32(4H, s, 6-aziridinyl), 1.91(3H, s, 5-methyl); IR(KBr pellet) 3512, 3384, 2959, 2926, 1678, 1622, 1514, 1467, 1381, 1235, 1017, 992, 810 cm⁻¹; MS(EI)276(M⁺), 258(M⁺-H₂O), 243, 230, 215, 202, 188, 174, 160, 145, 132, 122, 108. Anal. Calcd(C₁₄H₁₆N₂O₄)C, H, N.

Kinetics Studies using Recombinant DT-diaphorase. Kinetics studies were carried out in 0.05 M pH 7.4 Tris buffer under anaerobic conditions, employing Thunberg cuvettes. A 4.0 mM of quinone stock solution was prepared in dimethyl sulfoxide(DMSO). To the top port of the cuvette was added the quinone stock solution, and to the bottom port was added the recombinant DT-diaphorase and NADH stock solutions in Tris buffer. Both portions were purged with argon for 20 min and equilibrated at 30° C. for 10 min. The ports were then mixed and the reaction was followed at 336 nm for 10 min to obtain initial rates. The final concentrations of the mixture were 0.3 mM NADH, 1.3×10⁻⁶ to 6.7×10⁻⁵ M of quinone substrate and 1.4×10⁻⁹ M of recombinant DT-diaphorase. To calculate Vₘₐₓ, the value of □□ must be obtained. □□ was calculated from the initial and final absorbances for complete quinone reduction; all values are between 8000 to 9000 M⁻¹ cm⁻¹. All data were fitted to a Lineweaver-Burke plot from which V_{MAX} and K_{M} were obtained.

Modeling into the DT-diaphorase Active Site. Crystallographic coordinates for human DT-diaphorase (1D4A) were obtained from Protein Data Bank. The coordinates were used as downloaded from Protein Data Bank and are unrefined. For modeling purposes, INSIGHT II from Molecular Simulations, Inc. (San Diego) was used as previously described. {Huang, X., Suleman, A., and Skibo, E. B., Rational Design of Pyrrolo[1,2-a ]benzimidazole, Based Antitumor Agents Targeting the DNA Major Groove, Bioorg. Chem., 2000, Accepted December Issue; Suleman, A. and Skibo, E. B., Insights into the Mechanism and Substrate Specificty of Human DT-Diaphorase through Molecular Modeling, Biochemistry, 2000}.

Alkylation of DNA by Reduced Indoloquinones. To a mixture of 1-2 mg of sonicated (600 bp) calf thymus DNA in 2.0 mL of 0.05 M of pH 7.4 tris buffer and 2 mg of Pd on carbon was added a five-to-one base pair equivalent amount of the indoloquinone dissolved in 0.5 mL of dimethylsulfoxide. The resulting solution was degassed under argon for 30 min., after which the mixture was purged with H₂ for 10 min. The solution was then purged with argon for 10 min. and placed in a 30° C. bath for 24 h. The reaction was opened to the air and the catalyst was removed with a Millex-PF 0.8 μM syringe filter. The filtrate was adjusted to 0.3 M acetate with a 3 M stock solution of pH 5.1 acetate and then diluted with two volumes of ethanol. The mixture was chilled at −20° C. for 12 h and the DNA pellet collected by centrifuging at 12,000 g for 20 min. The pellet was redissolved in water and then precipitated and centrifuged again. The resulting blue or red pellet was suspended in ethanol, centrifuged, and dried. The dried pellet was weighed and dissolved in 1 mL of double distilled water resulting in a clear colored solution with □ₘₐₓ˜550 nm, □˜750 M⁻¹ cm⁻¹. This is the chromophore of the aminoquinone resulting from nucleophile-mediated opening of the aziridine ring. Model 2′-chloroethyl aminoquinones for extinction coefficient determination were prepared by treatment of the indoloquinone with HCl. {Skibo, E. B. and Xing, C., Chemistry and DNA Alkylation Reactions of Aziridinyl Quinones: Development of an Efficient Alkylating Agent of the Phosphate Backbone, Biochemistry, 1998, 37, 15199-15213}.

In vivo Evaluation. The B-16 melanoma in C57/bl mice syngraft model was employed to determine in vivo activity. {Griswold, D. P., Consideration of the Subcutaneously Implanted B16 Melanoma as a Screening Model for Potential Anticancer Agents, Cancer Chemother Reports, 1972, 3, 315-324}. Each agent was evaluated at three doses: 2, 3 or 5 mg/kg/day, on days 1, 5, and 9 after subcutaneous tumor implantation of 10⁵ cells in the front flank on day 0. “Toxic” means that there was early lethality, or ≧50% lethality prior to any deaths in the control group. The treated over control values (T/C) were measured at day 25 of the study. A T/C value<40% is considered active. NA means that the compound was not active. The control was obtained with drug-free animals.

| | | Calculated | | | Found | | |
|---|---|---|---|---|---|---|---|
| Compound # | (Formula) | C % | H % | N % | C % | H % | N % |
| 10a | C₁₂H₁₁NO₂ | 71.62 | 5.51 | 6.96 | 71.65 | 5.54 | 6.89 |
| 10c | C₁₂H₁₁NO₂ | 71.62 | 5.51 | 6.96 | 71.66 | 5.48 | 7.01 |
| 10d | C₁₃H₁₃NO₂ | 72.54 | 6.09 | 6.51 | 72.75 | 6.21 | 6.34 |
| 11a | C₁₂H₁₀N₂O₄ | 58.53 | 4.09 | 11.38 | 58.35 | 4.10 | 11.30 |
| 11c | C₁₂H₁₀N₂O₄ | 58.53 | 4.09 | 11.38 | 58.58 | 4.08 | 11.37 |
| 11d | C₁₃H₁₂N₂O₄ | 59.99 | 4.65 | 10.77 | 59.93 | 4.65 | 10.70 |
| 12a | C₁₂H₁₂N₂O₄ | 58.06 | 4.87 | 11.29 | 58.03 | 4.92 | 11.20 |
| 12c | C₁₂H₁₂N₂O₄ | 58.06 | 4.87 | 11.29 | 58.05 | 4.86 | 11.17 |
| 12d | C₁₃H₁₄N₂O₄ | 59.53 | 5.38 | 10.68 | 59.55 | 5.37 | 10.55 |
| 13a | C₁₂H₁₁NO₄ | 61.80 | 4.75 | 6.01 | 61.81 | 4.74 | 5.93 |
| 13c | C₁₂H₁₁NO₄ | 61.80 | 4.75 | 6.01 | 61.91 | 4.79 | 5.96 |
| 13d | C₁₃H₁₃NO₄ | 63.14 | 5.30 | 5.67 | 63.15 | 5.28 | 5.61 |
| 1a | C₁₃H₁₂N₂O₃ | 63.92 | 4.95 | 11.47 | 63.93 | 4.99 | 11.43 |
| 2a | C₁₄H₁₄N₂O₃ | 65.10 | 5.46 | 10.85 | 65.16 | 5.44 | 10.89 |
| 3a | C₁₃H₁₂N₂O₃ | 63.92 | 4.95 | 11.47 | 63.92 | 4.91 | 11.42 |
| 1b | C₁₅H₁₄N₂O₄ | 62.93 | 4.93 | 9.78 | 62.86 | 4.94 | 9.73 |
| 2b | C₁₆H₁₆N₂O₄ | 63.99 | 5.37 | 9.33 | 63.87 | 5.39 | 9.31 |
| 3b | C₁₅H₁₄N₂O₄ | 62.93 | 4.93 | 9.78 | 63.00 | 4.93 | 9.82 |
| 1c | C₁₇H₁₆N₂O₅ | 62.19 | 4.91 | 8.53 | 61.22 | 4.93 | 8.58 |
| 2c | C₁₈H₁₈N₂O₅ | 63.15 | 5.30 | 8.18 | 63.13 | 5.32 | 8.19 |
| 3c | C₁₇H₁₆N₂O₅ | 62.19 | 4.91 | 8.53 | 62.16 | 4.88 | 8.50 |
| 14 | C₁₃H₁₂N₂O₃ | 63.92 | 4.95 | 11.47 | 64.04 | 4.93 | 11.39 |
| 15 | C₁₃H₁₄N₂O₃ | 63.40 | 5.73 | 11.38 | 63.28 | 5.78 | 11.33 |
| 16 | C₁₃H₁₃NO₃ | 67.52 | 5.67 | 6.06 | 67.40 | 5.73 | 5.99 |
| 5a | C₁₅H₁₆N₂O₃ | 66.16 | 5.92 | 10.29 | 66.05 | 6.01 | 10.18 |
| 5b | C₁₇H₁₈N₂O₄ | 64.95 | 5.77 | 8.91 | 64.72 | 5.65 | 8.86 |
| 17b | C₁₃H₁₅NO₃ | 66.93 | 6.48 | 6.01 | 66.71 | 6.43 | 6.02 |
| 18a | C₁₃H₁₃NO₄ | 63.15 | 5.30 | 5.67 | 63.04 | 5.30 | 5.67 |
| 18b | C₁₄H₁₅NO₄ | 64.36 | 5.79 | 5.36 | 64.51 | 5.82 | 5.37 |
| 18c | C₁₂H₁₁NO₄ | 61.80 | 4.75 | 6.01 | 61.77 | 4.82 | 5.95 |
| 19a | C₁₃H₁₂N₂O₆ | 53.42 | 4.14 | 9.59 | 53.38 | 4.21 | 9.73 |
| 19b | C₁₄H₁₄N₂O₆ | 54.90 | 4.61 | 9.15 | 54.88 | 4.60 | 9.13 |
| 19c | C₁₂H₁₀N₂O₆ | 51.80 | 3.62 | 10.07 | 51.74 | 3.70 | 10.06 |
| 20a | C₁₁H₁₂N₂O₅ | 52.38 | 4.80 | 11.11 | 52.67 | 4.76 | 11.03 |
| 20b | C₁₂H₁₄N₂O₅ | 54.13 | 5.30 | 10.52 | 54.06 | 5.33 | 10.48 |
| 20c | C₁₁H₁₂N₂O₅ | 52.38 | 4.80 | 11.11 | 52.32 | 4.82 | 11.10 |
| 21a | C₁₁H₁₁NO₅ | 55.69 | 4.67 | 5.91 | 55.44 | 4.64 | 5.89 |
| 21b | C₁₂H₁₃NO₅ | 57.27 | 5.22 | 5.58 | 57.14 | 5.29 | 5.58 |
| 21c | C₁₁H₁₁NO₅ | 55.69 | 4.67 | 5.91 | 55.64 | 4.68 | 5.85 |
| 6a | C₁₂H₁₂N₂O₄ | 58.06 | 4.87 | 11.29 | 57.99 | 4.89 | 11.23 |
| 7a | C₁₃H₁₄N₂O₄ | 59.53 | 5.38 | 10.68 | 59.50 | 5.40 | 10.57 |
| 9a | C₁₂H₁₂N₂O₄ | 58.06 | 4.87 | 11.29 | 58.20 | 4.90 | 11.13 |
| 6e | C₁₆H₁₆N₂O₆ | 57.83 | 4.85 | 8.43 | 57.95 | 4.81 | 8.38 |
| 22 | C₁₅H₁₆N₂O₆ | 56.22 | 5.03 | 8.74 | 56.24 | 5.01 | 8.69 |
| 23 | C₁₃H₁₆N₂O₅ | 55.70 | 5.75 | 10.00 | 55.54 | 5.83 | 9.93 |
| 24 | C₁₃H₁₅NO₅ | 58.86 | 5.70 | 5.28 | 58.79 | 5.70 | 5.31 |
| 7c | C₁₄H₁₆N₂O₄ | 60.86 | 5.84 | 10.14 | 60.81 | 5.84 | 10.09 |
| 25a | C₁₃H₁₄N₂O₆ | 53.06 | 4.80 | 9.52 | 53.09 | 4.81 | 9.53 |
| 25b | C₁₄H₁₆N₂O₆ | 54.54 | 5.23 | 9.09 | 54.47 | 5.21 | 9.10 |
| 25c | C₁₂H₁₂N₂O₆ | 51.43 | 4.32 | 10.00 | 51.51 | 4.31 | 10.02 |
| 26a | C₁₃H₁₃NO₆ | 55.91 | 4.69 | 5.02 | 55.83 | 4.71 | 5.02 |
| 26b | C₁₄H₁₅NO₆ | 57.33 | 5.16 | 4.78 | 57.26 | 5.20 | 4.69 |
| 26c | C₁₂H₁₁NO₆ | 54.34 | 4.18 | 5.28 | 54.40 | 4.23 | 5.32 |
| 6c | C₁₄H₁₄N₂O₅ | 57.93 | 4.86 | 9.65 | 57.84 | 4.83 | 9.59 |
| 7b | C₁₅H₁₆N₂O₅ | 59.20 | 5.30 | 9.21 | 59.07 | 5.25 | 9.20 |
| 9b | C₁₃H₁₂N₂O₅ | 56.52 | 4.38 | 10.14 | 56.55 | 4.39 | 10.09 |
| 6d | C₁₆H₁₆N₂O₆ | 57.83 | 4.85 | 8.43 | 57.74 | 4.91 | 8.40 |
| 27a | C₁₃H₁₄N₂O₅ | 56.1 | 5.07 | 10.07 | 56.01 | 5.04 | 9.98 |
| 28a | C₁₄H₁₆N₂O₅ | 57.5 | 5.52 | 9.59 | 57.4 | 5.58 | 9.54 |
| 28b | C₁₄H₁₆N₂O₄ | 60.9 | 5.84 | 10.14 | 60.7 | 5.86 | 10.09 |
| 29a | C₁₄H₁₆N₂O₆ | 57.6 | 5.64 | 11.2 | 57.34 | 5.69 | 10.96 |
| 29b | C₁₃H₁₄N₂O₅ | 56.1 | 5.07 | 10.07 | 55.9 | 5.01 | 9.98 |
| 29c | C₁₄H₁₆N₂O₅ | 57.5 | 5.52 | 9.59 | 57.46 | 5.51 | 9.56 |
| 30a | C₁₂H₁₄N₂O₅ | 54.1 | 5.30 | 10.5 | 53.97 | 5.37 | 10.48 |
| 30b | C₁₁H₁₂N₂O₄ | 55.9 | 5.12 | 11.86 | 55.7 | 5.08 | 11.62 |
| 30c | C₁₂H₁₄N₂O₄ | 57.6 | 5.64 | 11.2 | 57.34 | 5.69 | 10.96 |
| 31a | C₁₂H₁₃NO₅ | 57.4 | 5.22 | 5.58 | 57.33 | 5.26 | 5.49 |
| 31b | C₁₁H₁₁NO₄ | 59.7 | 5.01 | 6.33 | 59.2 | 5.03 | 6.48 |
| 31c | C₁₂H₁₃NO₄ | 61.3 | 5.57 | 5.95 | 61.1 | 5.57 | 5.96 |
| 6b | C₁₃H₁₄N₂O₄ | 59.5 | 5.38 | 10.68 | 59.43 | 5.39 | 10.66 |
| 8a | C₁₃H₁₄N₂O₄ | 59.5 | 5.38 | 10.68 | 59.2 | 5.40 | 10.52 |
| 8b | C₁₇H₁₈N₂O₆ | 58.95 | 5.24 | 8.09 | 59.02 | 5.33 | 8.02 |
| 8c | C₁₄H₁₆N₂O₄ | 60.86 | 5.84 | 10.1 | 60.65 | 5.78 | 9.97 |

What we claim is

1. A cyclopent[b]indole-based aziridinyl quinone having the structure: 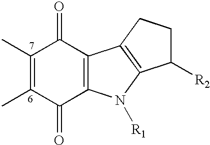
wherein positions 6 and 7 are each substituted with a substituent selected from aziridine, hydrogen or methyl, provided that positions 6 and 7 are not substituted with the same group; and wherein R₁ and R₂ are selected from the group consisting of hydrogen, methyl, hydroxyl or acetate:

2. An indole-based aziridinyl quinone having the structure: 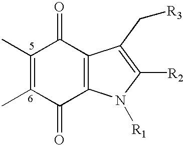
wherein positions 5 and 6 are each substituted with a substituent selected from aziridine, hydrogen or methyl, provided that positions 5 and 6 are not substituted with the same group; and wherein R₁ is selected from the group consisting of hydrogen and methyl; R₂ is selected from the group consisting of CO₂Et, CH₂OH, and CH₂OAc; and R₃ is selected from the group consisting of COH, CH₂OH, and CH₂OAc.

3. A cyclopent[b]indole-based aziridinyl quinone as set forth in claim 1 having the structure:

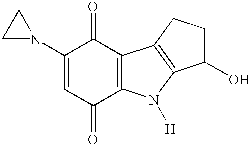

4. A cyclopent[b]indole-based aziridinyl quinone as set forth in claim 1 having the structure:

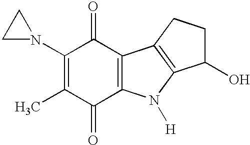

5. A cyclopent[b]indole-based aziridinyl quinone as set forth in claim 1 having the structure:

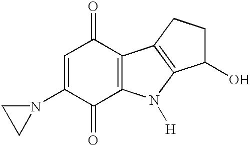

6. A cyclopent[b]indole-based aziridinyl quinone as set forth in claim 1 having the structure:

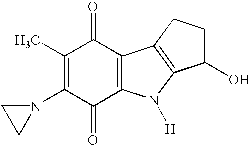

7. A cyclopent[b]indole-based aziridinyl quinone as set forth in claim 1 having the structure:

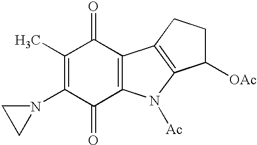

8. An indole-based aziridinyl quinone as set forth in claim 2 having the structure:

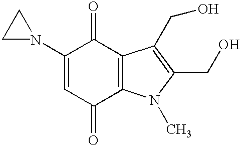

9. An indole-based aziridinyl quinone as set forth in claim 2 having the structure:

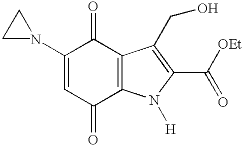

10. An indole-based aziridinyl quinone as set forth in claim 2 having the structure:

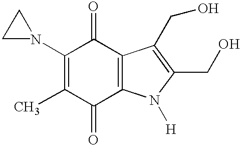

11. An indole-based aziridinyl quinone as set forth in claim 2 having the structure:

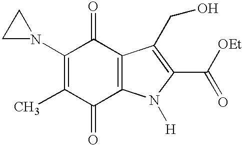

12. An indole-based aziridinyl quinone as set forth in claim 2 having the structure:

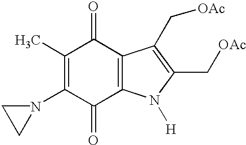

13. An indole-based aziridinyl quinone as set forth in claim 2 having the structure:

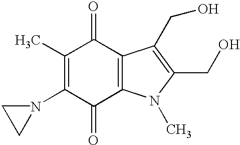

14. A method for treating cancer in an animal or human subject comprising administering to the subject a pharmaceutically effective amount of the compound of claim 1.

15. A method for treating cancer in an animal or human subject comprising administering to the subject a pharmaceutically effective amount of the compound of claim 2.

16. A method for treating an animal or human subject having a histological cancer type possessing high levels of DT-diaphorase, comprising administering to the subject a chemical substrate having a substrate specificity (V_{MAX}/K_{M}) of less than about 10×10⁻⁴s⁻¹.
